SEQUENCE LISTING (19 sequences)

SEQ ID NO: 1 - DNA, 24 bp, Artificial Sequence
  probe
gataggaattattactgcccttcc

SEQ ID NO: 2 - DNA, 21 bp, Artificial Sequence
  probe
gacaagaaagcactgttgacc

SEQ ID NO: 3 - DNA, 24 bp, Artificial Sequence
  probe
gataggaattattactgcccttcc

SEQ ID NO: 4 - DNA, 23 bp, Artificial Sequence
  probe
gctaccaatcagcttcttagccc

SEQ ID NO: 5 - DNA, 30 bp, Artificial Sequence
  probe
ccaaaatttgcctgcattaccggtcgatgc

SEQ ID NO: 6 - DNA, 20 bp, Artificial Sequence
  probe
atccaggttacggatatagt

SEQ ID NO: 7 - DNA, 21 bp, Artificial Sequence
  probe
cggtgatggtgctgcgttgga

SEQ ID NO: 8 - DNA, 21 bp, Artificial Sequence
  probe
accaccgcacgatagagattc

SEQ ID NO: 9 - DNA, 21 bp, Artificial Sequence
  probe
gcgaagagtttgtcctcaacc

SEQ ID NO: 10 - DNA, 21 bp, Artificial Sequence
  probe
ggagcgggagaaatggatatg

SEQ ID NO: 11 - DNA, 21 bp, Artificial Sequence
  probe
aaagtcgctctgagttgttat

SEQ ID NO: 12 - DNA, 20 bp, Artificial Sequence
  forward probe
cgagtccctccttcgtatgg

SEQ ID NO: 13 - DNA, 18 bp, Artificial Sequence
  reverse probe
gctctggccgctcaatca

SEQ ID NO: 14 - DNA, 21 bp, Artificial Sequence
  reporter sequence probe
ctgatgacgtagaagagtaca

SEQ ID NO: 15 - RNA, 19 nt, Artificial Sequence
  siRNA oligo target sequence
agacagaccccaaacguaa

SEQ ID NO: 16 - RNA, 19 nt, Artificial Sequence
  siRNA oligo target sequence
gaaaauggcuugucgaauu

SEQ ID NO: 17 - RNA, 19 nt, Artificial Sequence
  siRNA oligo target sequence
agggaaacaucuauaacga

SEQ ID NO: 18 - RNA, 19 nt, Artificial Sequence
  siRNA oligo target sequence
ccgccaacaucacggucaa

SEQ ID NO: 19 - protein, 5 aa, Artificial Sequence
  peptide
LXXLL

(19) United States
(12) Patent Application Publication
(10) Pub. No.: US 2017/0027896 A1
(43) Pub. Date: Feb. 2, 2017

(54) METHODS AND COMPOSITIONS FOR THE TREATMENT OF VASCULAR MALFORMATION

(71) Applicants: IFOM - FONDAZIONE ISTITUTO FIRC DI ONCOLOGIA MOLECOLARE, Milano(MI) (IT); UNIVERSITA' DEGLI STUDI DI MILANO, Milano (IT)

(72) Inventors: Elisabetta DEJANA, Milano(MI) (IT); Maria Grazia LAMPUGNANI, Milano(MI) (IT)

(21) Appl. No.: 15/302,288

(22) PCT Filed: Apr. 10, 2015
(86) PCT No.: PCT/EP2015/057854

§ 371 Date: Oct. 6, 2016

(30) Foreign Application Priority Data
Apr. 10, 2014 (EP) 14164118.3

(51) Int. Cl. A61K 31/192; A61K 31/12; A61K 31/05; A61K 38/00; A61K 31/7088; A61K 9/51; A61K 45/06; A61K 31/357; A61K 31/35

(52) U.S. Cl. CPC A61K 31/192; A61K 31/357; A61K 31/12; A61K 31/05; A61K 31/35; A61K 31/7088; A61K 9/51; A61K 45/06; A61K 38/00

(57) ABSTRACT

An inhibitor of Wnt/β-catenin signaling for use in the treatment and/or prevention of a pathology having vascular malformation is provided. The inhibitor may be a small molecule, a protein, a peptide or an antisense nucleic acid. The invention also relates to pharmaceutical compositions and to method of treatment.

### FIELD OF THE INVENTION

The invention relates to a Wnt/beta-catenin signaling inhibitor for the treatment of a pathology characterized by vascular malformation, in particular characterized by Endothelial-to-Mesenchymal Transition, particularly Cerebral Cavernous Malformation. The inhibitor may be a small molecule, a protein, a peptide or an antisense nucleic acid. The invention also relates to pharmaceutical compositions and to method of treatment.

### BACKGROUND OF THE INVENTION

Vascular malformations that characterize the disease known as Cerebral Cavernous Malformation (CCM) are mainly concentrated in the central nervous system, and they typically show multiple lumens and vascular leakage{Clatterbuck, 2001 #17; Wong, 2000 #18}. Such organ location of pathological vessels can result in several neurological symptoms, including hemorrhagic stroke³. In human, mutations in any one of the three independent genes known as CCM1, CCM2 and CCM3 have been linked to the familial variant of CCM⁵. In murine models, inducible endothelium-specific loss-of-function mutations of any of the CCM genes^{6,7} in newborns can reproduce the cerebral vascular phenotype. In addition, constitutive endothelial-selective inactivation of CCM3 is embryonically lethal for general problems of vascular development⁸. Although neural-specific mutation of CCM3 in rare cases may induce a cerebral vascular phenotype⁹, these data strongly suggest that mutations of CCM genes in endothelial cells contribute to CCM pathological phenotype. However, the mechanisms of action of these genes in endothelial cells are still largely unknown. Up to date the only therapy for CCM disease is surgery⁴.

It has been reported that knocking down the CCM1 protein in cultured aortic endothelial cells promotes Wnt/b-catenin signaling¹⁹. However, to date, there has been no indication of the relevance of the b-catenin pathway in the formation of vascular lesions in vivo. Some studies, have shown that during embryo development activation of canonical Wnt/b-catenin signaling is required for brain angiogenesis and for the correct differentiation of the blood-brain barrier microvasculature¹¹⁻¹³. These effects need to be tightly regulated in the adult to avoid uncontrolled vascular proliferation. Indeed, in physiological conditions b-catenin signaling sharply declines postnatally and is essentially undetectable in the adult¹³.

WO2009148709 disclosed compositions and methods for decreasing vascular permeability in a blood vessel and treating or preventing conditions associated with defects or injuries of vascular endothelium. For example, the disclosed compositions and methods can be used to treat a vascular dysplasia such as cerebral cavernous malformation (CCM). These methods relate generally to the use of compositions that inhibit RhoA GTPase levels or activity, such as inhibitors of 3-hydroxy-3 methylglutaryl-coenzyme A (HMG-CoA) reductase. The application provides, as examples of RhoA GTPase inhibitor, a statin molecule, such as Simvastatin, or nitrogen-containing biphosphonate such as Pamidronate, Neridronate, Olpadronate, Alendronate, Ibandronate, Risedronate, and Zoledronate.

In WO2012135650, derivatives of sulindac that lack cyclooxygenase inhibitory activity are provided along with pharmaceutical compositions containing them and use for treatment or prevention of cancer. The derivatives of sulindac are also suitable for treating chronic inflammatory conditions. A method for preparing the derivatives is also provided. It is also claimed their use in Alzheimer disease.

Kundu J K et al. [“Beta-catenin-mediated signaling: a novel molecular target for chemoprevention with anti-inflammatory substances” Biochimica et Biophysica Acta 1765 (2006): 14-24] focus on beta-catenin-mediated signaling pathways, particularly in relation to its contribution to carcinogenesis, and the modulation of inappropriately activated beta-catenin-mediated signaling by non-steroidal anti-inflammatory drugs and chemopreventive phytochemicals possessing anti-inflammatory properties. In Table 1 and 2, Kundu et al list all substances able to modulate the beta-catenin mediated signaling pathways, being both nonsteroidal anti-inflammatory agents (NSAIDs) and anti-inflammatory phytochemicals. Other beta-catenin inhibitors and their mechanism of action are described in Anastas and Moon, 2013, Nature Rev-Cancer, 13, 11-26; Rosenbluh et al, 2014, Trends Pharmac. Sci 35, 103-109; Takahashi-Yanaga and Kahn, 2010, Olin Cancer Res 16, 3153-3162.

In McDonald D A et al (“Fasudil decreases lesion burden in murine model of cerebral cavernous malformation”, Stroke 43, 571-574. 2012), a model of CCM1 disease was treated with the Rho kinase inhibitor, fasudil.

However, there is still the need for a pharmacological treatment of CCM since at present the disease may be only surgically treated.

### SUMMARY OF INVENTION

In the present invention, the inventors have found that alterations in the transcription activity of beta-catenin (b-catenin or β-catenin) in endothelial cells contribute to the pathological phenotype of CCM in vivo. Here they report that the CCM3 protein is indeed a regulator of b-catenin transcription activity in endothelial cells both in vitro and in vivo. Furthermore, the inventors have surprisingly found that agents able to reduce b-catenin-mediated transcription activity are able to reduce the number and extension of brain or retina vascular malformations and to prevent the appearance of new vascular lesions.

In the present invention it was surprisingly found that compounds able to reduce β-catenin-mediated activity such as the NSAIDs sulindac sulfide and sulindac sulfone, significantly reduce the number and dimension of vascular lesions in the central nervous system of mice with endothelial-cell-specific CCM3 knockout gene, a model of Cerebral cavernous malformation (CCM). CCM is a vascular disease that affects blood vessels in the central nervous system, which become malformed, leaky and prone to hemorrhage. The organ location is critical, both for neurological consequences and therapeutic intervention, which is exclusively surgical to date. Thus, compounds able to reduce β-catenin-mediated activity represent pharmacological tools for inhibition of the formation of vascular lesions, particularly with a view to patients affected by the familial variant of CCM, who continue to develop new malformations over time.

The results shown in the present invention are applicable to any pathology characterized by vascular malformation. Such pathology show dismantling of cell-to-cell junction and expression of EndMT (Endothelial-to-Mesenchymal Transition), markers (such as Klf4, Klf2, Ly6a, S100a4, CD44, Id1, a-Sma, Slug, PAI1, N-cadherin, Zeb2, Fadini et al, 2012: Margariti et al, 2012; Li et al, 2012; Liang et al 2011; Stein et al, 2006; Medici et al, 2012) in endothelial cells.

The present invention is also based on the surprising finding that inhibition of the Wnt/β-catenin pathway, in particular of β-catenin signaling using for instance the NSAID, sulindac sulfone (exisulind) inhibits the expression of EndMT markers. Such markers are present in pathologies characterized by vascular malformation such as CCM.

In a first aspect, the invention provides an inhibitor of Wnt/β-catenin signaling for use in the treatment and/or prevention of a pathology characterized by vascular malformation. Preferably the inhibitor is a β-catenin inhibitor, in particular an inhibitor of β-catenin transcriptional signaling and/or an inhibitor of β-catenin nuclear translocation.

Still preferably the inhibitor is a small molecule inhibitor. In a preferred embodiment the inhibitor is selected from the group consisting of: quercetin, ZTM000990, PKF118-310, PKF118-744, PKF115-584, PKF-222-815, CPG049090, PNU-74654, ICG-001, NSC668036, N′-[(E)-(5-methyl-2-furyl)methylidene]-2-phenoxybenzohydrazide, N′-[(E)-1-(5-methyl-2-thienyl)ethylidene]-2-phenoxyacetohydrazide, 5-[2-(5-methyl-2-furyl)ethyl]-2-(2-thienyl)-1H-indole, 2-(2-furyl)-5-[(E)-2-(5-methyl-2-furyl)ethenyl]-1H-indole, N-[(E)-(5-methyl-2-furyl)methylidene]-4-(4-pyridinyl)-8-quinolin-amine, 2-(2-furyl)-5-[2-(5-methyl-2-furyl)ethyl]-1H-indole, 7-{(2E)-2-[(5-methyl-2-furyl)methylene]hydrazino}-N-(2-phenylethyl)-5,6-dihydrobenzo[h]isoquinoline-9-carboxamide, 1-{[(E)-(5-methyl-2-furyl)methylidene]amino}-3-(4-pyridinyl)-2,4-(1H,3H)-quinazolinedione, N-(5-methyl-2-furyl)-N-(2′-phenoxy[1,1′-biphenyl]-3-yl)amine, 4-{[7-(5-methyl-2-furyl)-2-naphthyl]oxy}pyridine, N-(5-bromo-1,3,4-oxadiazol-2-yl)-4-hydroxy-2-oxo-6-phenyl-2H-pyran-3-carboxamide, 4-hydroxy-N-(5-methyl-2-furyl)-2-oxo-6-phenyl-2H-pyran-3-carbox-amide, 3-[(E)-2-(5-bromo-1,3,4-thiadiazol-2-yl)ethenyl]-4-hydroxy-6-ph-enyl-2H-pyran-2-one, N-(5-bromo-1,3,4thiadiazol-2-yl)-4-hydroxy-2-oxo-6-phenyl-2H-pyran-3-carboxamide, 5-[(3-amino-1H-1,2,4-triazol-5-yl)methyl]-3-[3-fluoro-4-(4-morpholinyl)phenyl]-1,3-oxazolidin-2-one, 4-[(3-amino-1H-1,2,4-triazol-5-yl)methyl]-1-[3-fluoro-4-(4-morpholinyl)phenyl]-2-imidazolidinone, 1-benzhydryl-4-(5-bromo-2-furoyl)piperazine, 1-benzhydryl-4-[(5-methyl-2-thienyl)carbonyl]piperazine, benzyl (2E)-2-[1-(4-methyl-2-thienyl)ethylidene]hydrazinecarboxylate, 2-(4-chlorophenyl)-6-methyl-5-(5-methyl-1,3,4-oxadiazol-2-yl)[1,3]thiazolo[3,2-b][1,2,4]triazole, N-(5-methyl-3-isoxazolyl)-N′-[(5-phenyl-1,3,4-oxadiazol-2-yl)carbonyl]urea, N-[3-(2-{[(5-chloro-2-thienyl)methyl]sulfonyl}hydrazino)-3-oxopropyl]benzenesulfonamide-5-[3-(4-phenoxyphenyl)propyl]-1,3,4-oxadiazol-2-ol, N-(3-methyl-5-isoxazolyl)-4-phenoxybenzamide, 4-hydroxy-N-(3-methyl-5-isoxazolyl)-2-oxo-6-phenoxy-2H-pyran-3-carboxamide, 2-phenoxy-N′-[(Z)-phenyl(2-thienyl)methylidene]benzo-hydrazide, 2-anilino-N′-[(Z)-2-furyl(phenyl)methylidene]benzohydrazide, 4-[(Z)-1-(3-methyl-5-isoxazolyl)-2-phenylethenyl]phenyl 2-(1-pyrrolidinyl)ethyl ether, 5-methyl-2-furaldehyde[(3Z)-2-oxo-1-(4-pyridinyl)-1,2-dihydro-3H-indol-3-ylidene]hydrazone, (2Z)—N-[(5-methyl-2-furyl)methyl]-2-[2-oxo-1-(4-pyridinyl)-1,2-dihydro-3H-indol-3-ylidene]ethanamide, (2Z)—N-[(3-methyl-5-isoxazolyl)methyl]-2-[2-oxo-1-(4-pyridinyl)-1,2-dihydro-3H-indol-3-ylidene]ethanamide, (2-chloro-1,3-thiazol-5-yl)methyl 4-(4-morpholinylsulfonyl)phenyl ether, N-(4,5-dihydronaphtho[1,2-d][1,3]thiazol-2-yl)-N-(4-phenoxybutyl)methanesulfonamide, N-(6-methoxy-4,5-dihydronaphtho[1,2-d][1,3]thiazol-2-yl)-N-[2-(1-methyl-3-phenylpropoxy)ethyl]acetamide, 4-{2-[(5-methyl-2-furyl)methoxy]benzylidene}-1-(4-pyridinylsulfonyl)piperidine, 4-{2-[(5-bromo-2-furyl)methoxy]benzylidene}-1-isonicotinoylpiperidine, N-(4,5-dihydronaphtho[1,2-d][1,3]thiazol-2-yl)-N-(4-phenylpentyl)acetamide, N-(4,5-dihydro-3H-naphtho[1,2-d]imidazol-2-yl)-N-[2-(2-phenylethoxy)ethyl]methane sulfonamide, N′-[(Z)-(5-methyl-2-furyl)(2-pyridinyl)methylidene]-2-phenoxybenzohydrazide, sulindac sulfide, sulindac sulfone and their pharmaceutically acceptable salts, hydroxymatairesinol, hexachlorophene, a PPARγ agonist, or PPARγ-inactive analog, silibinin, milk thistle extract (cardio mariano), EGCG (epigallocatechin-3-gallate), White tea/Green tea, Sulforaphane, Resveratrol, Curcumin, Indole-3-carbinol, Ursolic acid, Docosahexanoic acid, Genistein, β-Lapachone and compounds listed in Table I [http://web.stanford.edu/group/nusselab/cgi-bin/wnt/].

Table I lists compounds that have been reported to inhibit Wnt signaling by targeting various components of the pathway, resulting in its inhibition. See Dodge, Annu Rev Pharmacol Toxicol. 2011; 51:289-310, Chen, Am J Physiol Gastrointest Liver Physiol. 2010 August; 299(2):G293-300, Barker Nat Rev Drug Discov. 2006 December; 5(12):997-1014, for reviews. These inhibitors all form part of the present invention.

TABLE I
Wnt/b-catenin signaling inhibitors

| | | Inhibitor/activator | Effect on | |
|---|---|---|---|---|
| Compound | Target | of the target | signaling | reference |
| Ant1.4Br/Ant | Wnt | Inhibitor (free | inhibits | Morrell 2008 |
| 1.4Cl | | Wnt, not | | |
| | | membrane- | | |
| | | bound Wnt) | | |
| Niclosamide | Frizzled | Inhibitor | inhibits | Chen 2009 |
| apicularen and | Vacuolar | Inhibitor | inhibits | Cruciat 2010 |
| bafilomycin | ATPase | | | |
| XAV939 | tankyrase | Activates Axin | inhibits | Huang, 2009 |
| | 1/Axin | | | |
| IWR | Axin | Activates Axin | inhibits | Chen et al |
| | | | | (2009) |
| pyrvinium | CK1 | Inhibitor | inhibits | Thorne |
| | | | | (2010) |
| NSC668036 | Dsh | Inhibitor | inhibits | Shan et al |
| | | | | (2005) |
| 2,4-diamino- | TCF/beta- | Inhibitor | inhibits | Chen 2009 |
| quinazoline | catenin | | | |
| Quercetin | TCF | Inhibitor | inhibits | Park et al |
| | | | | (2005) |
| ICG-001 | CREB- | Inhibitor | inhibits | Emami 2004 |
| | binding | | | |
| | protein | | | |
| PKF115-584 | TCF/beta- | Inhibitor | inhibits | Lepourcelet |
| (and several | catenin | | | et al (2004) |
| other | | | | |
| compounds) | | | | |

Still preferably the inhibitor is sulindac or sulindac sulfide or sulindac sulfone or an analog or a derivative thereof.

Sulindac sulfone is also named exisulind, Aptosyn®, fgn1 or Prevatac®.

The inhibitor may be an inhibitor of PDE5 (phosphodiesterase5) or an activator of PKG (protein kinase G or cyclicGMP-dependent protein kinase). PDE5 and PKG are direct and indirect (through regulation of cyclicGMP level) targets, respectively, of both sulindac sulfide and exisulind and contribute to regulate the phosphorylation of beta-catenin and to inhibit beta-catenin-driven signaling pathway (Tinsley et al, 2011; Thompson et al, 2000; Li et al, 2001).

Yet preferably the inhibitor is selected from the group consisting of: silibinin, EGCG (epigallocatechin-3-gallate), White tea/Green tea, Sulforaphane, Resveratrol, Curcumin, Indole-3-carbinol, Ursolic acid, Docosahexanoic acid, Genistein and β-Lapachone.

In a preferred embodiment the Wnt/β-catenin signaling inhibitor is a protein or peptide. Preferably the protein or peptide is administered directly or expressed via an administered expression system.

Still preferably the protein or peptide is Chibby, Axin, HDPR1, ICAT, or a fusion protein comprising an LXXLL peptide (SEQ ID NO: 19), DKK1, an antibody against frizzled such as OMP-18R5.

DKK1 (Dickkopf, Dkk) is a negative regulator of Wnt signaling (Glinka, 1998; Niehrs, 1999) The Dkk protein is secreted and rich in cysteines. Dkk does not bind to Wnt but interacts with the Wnt co-receptor LRP. An antibody to frizzled, such as OMP-18R5 can be used therapeutically and in the lab. It interacts with multiple Frizzled receptors (Gurney et al, Proc Natl Acad Sci USA. 2012 Jul. 17; 109(29):11717-22).

Yet preferably the Wnt/β-catenin signaling inhibitor is an antisense nucleic acid molecule. Still preferably a full-length antisense beta-catenin construct, beta-catenin siRNA, or beta-catenin shRNA.

In a preferred embodiment the inhibitor is expressed by a recombinant expression system suitable for administration to the subject.

In a preferred embodiment the recombinant expression system comprises an endothelium or a brain endothelium specific promoter element and, optionally, an inducer/repressor element.

In a preferred embodiment the inhibitor is encapsulated in nanoparticles, preferably the nanoparticles are engineered to target pathological endothelial cells.

In a still preferred embodiment the vascular malformation is characterized by endothelial-to-mesenchymal transition or the vascular malformation is associated with endothelial-to-mesenchymal transition (Medici et al, 2012; Fadini et al, 2012). In the vascular malformation of the invention, endothelial-to-mesenchymal transition is present.

Preferably the vascular malformation is within the central nervous system and/or the retina vasculature.

Still preferably the pathology is selected from the group consisting of: fibrodysplasia (Medici D et al. Nature Medicine 16:1400, 2010) ossificans progressive, cardiac fibrosis, kidney fibrosis, pulmonary fibrosis (Medici D and Kalluri R Semin Cancer Biol 22:379, 2012) and cerebral cavernous malformation.

Yet preferably the pathology is cerebral cavernous malformation.

In a preferred embodiment the cerebral cavernous malformation is caused by loss-of-function mutations in at least one of the genes selected from the group of: CCM1 (KRIT1), CCM2 (OSM) or CCM3 (PDCD10).

Still preferably the cerebral cavernous malformation is sporadic or familial.

It is a further object of the invention a pharmaceutical composition comprising an effective amount of at least one inhibitor as defined above and pharmaceutical acceptable vehicle for use in the treatment and/or prevention of a pathology characterized by vascular malformation.

Preferably the pharmaceutical composition further comprises an effective amount of at least another therapeutic agent.

In a preferred embodiment the other therapeutic agent is selected from the group of: anti-oxidant, TGF-β signaling pathway inhibitors, BMP signaling pathway inhibitors, VEGF signaling pathway inhibitors, Yap signaling pathway inhibitors, statins (see for example Hwang et al, 2013, Int J. Oncol 43, 261-270) and inhibitors of RhoA GTPase levels and/or activity.

In a preferred embodiment the pharmaceutical acceptable vehicle is a nanoparticle, preferably the nanoparticle is engineered to target pathological endothelial cells.

It is a further object of the invention a method of treating and/or preventing of a pathology characterized by vascular malformation comprising administering to a subject in need thereof an effective amount of an inhibitor of wnt/beta-catenin signalling.

These compounds can be administered by different routes, including orally, and can be given in dosages that are safe and effective in reducing vascular malformations and preventing the appearance of new vascular lesions in CCM (cerebral cavernous malformation, sporadic or familial form) patients.

In the present invention an inhibitor of Wnt/beta-catenin signaling is a chemical tool that as a final result of its action inhibits the transcriptional responses driven by beta-catenin. Target of such inhibitor can be any step and molecular component of the Wnt/beta-catenin signaling pathway.

In particular an inhibitor of Wnt/beta-catenin signaling is a beta-catenin inhibitor. A beta-catenin inhibitor is: an inhibitor of beta-catenin activity and/or signaling. The inhibitor may inhibit beta-catenin activity directly or indirectly by a) acting on beta-catenin, b) by promoting beta-catenin degradation, c) by interfering with the expression of beta-catenin, d) by competing with other agents for binding with beta-catenin. The inhibitor may be an inhibitor of beta-catenin mediated transcription and/or other levels of beta-catenin activity. The inhibitors are inhibitors of b-catenin transcription signaling. The inhibitor may also inhibit or prevent nuclear accumulation of active b-catenin.

In the present invention there are various ways to specifically inhibit Wnt signaling, in cell culture and/or in a subject. a) to use RNAi targeting various component of the pathway, such as LRP/Arrow, Dishevelled, this has been shown to work very well for Drosophila S2 cells (Matsubayashi 2004, Gong et al, 2004) but also in mammalian cells (Lu et al, 2004). b) There is a variety of small molecules that have been shown to inhibit Wnt signaling to various degrees. See Table I and their targets. One of these, IWP, has been shown to be effective in blocking Wnt secretion through inhibiting porcupine (Chen et al, 2009). c) The (secreted) Wnt signal can be blocked by an excess of the ligand binding domain of its receptor, Frizzled. This domain is best made as its natural fusion in the FRP/Frz form. Alternatively, it can be expressed on the surface of target cells using a GPI anchor, which works well (Cadigan, 1998). d) Another way of inhibiting Wnt is to add excess of Dickkopf (Dkk) protein (Glinka, 1998). This works well in cell culture and in vivo. Dkk binds to the LRP co-receptor for Wnt. e) To block signaling inside cells, several workers have used dominant negative Dishevelled (Wallingford 2000). f) overexpressing intact Axin, a negative regulator of the Wnt pathway, works very well (Zeng, 1997, Itoh 1998; Willert, 1999). g) Overexpressing full length GSK can also block Wnt signaling effectively (He, 1995). h) There are dominant negative forms of TCF that can be used to block Wnt signaling in the nucleus (Molenaar 1996). i) An antibody to frizzled, OMP-18R5 can be used therapeutically and in the lab. It interacts with multiple Frizzled receptors (Gurney et al. 2012).

In the present invention a pathology characterized by vascular malformation presents vessels of any type and district with localized abnormal organization, in which endothelial cells show disordered cell-to-cell contacts and/or expression of endothelial-to-mesenchymal transition (EndMT) markers (such as Klf4, Klf2, Ly6a, S100a4, CD44, Id1, a-Sma, Slug, PAI1, N-cadherin, Zeb2, Fadini et al, 2012: Margariti et al, 2012; Li et al, 2012; Liang et al 2011; Stein et al, 2006; Medici et al, 2012) and/or impaired barrier function. Association of mural cells, such pericytes can also be impaired. Examples of such malformations are found in the Cerebral Cavernous Malformation (CCM) pathology.

In the present invention a vascular malformation characterized by EndMT is a local aberration of the vessels in which endothelial cells have lost endothelial differentiation. As a consequence, the function of the endothelial layer is impaired and the area of vessel affected by such abnormality is structurally abnormal, hyper-permeable, inflamed and prone to hemorrhage.

According to this aspect of the invention, a treatment and/or prevention of a pathology characterized by vascular malformation can be effective to mitigate at least one symptom of vascular malformation, in particular a vascular malformation characterized by EndMT particularly CCM.

When treating the underlying cause of a pathology characterized by vascular malformation, it is believed that management of symptoms can likewise be achieved. By management of symptoms, it is intended that the severity of symptoms can be maintained (i.e., worsening or advancement of symptoms is controlled) or, more preferably, the severity of symptoms can be reduced either in whole or in part.

The symptoms include abnormal clusters of dilated blood vessels, seizures, stroke symptoms, hemorrhages and headache, lesions. Symptoms typically depend on the location of the malformation and may include: Seizures ranging in severity, duration and intensity, Neurological deficits, such as weakness in arms and legs as well as problems with vision, balance, memory and attention, Headaches ranging in severity, duration and intensity, Bleeding, called a hemorrhage, in the brain that may damage surrounding brain tissue.

Any of one or more different inhibitors of beta-catenin can be used, as well as combinations thereof. These can include, without limitation, small molecule inhibitors, protein and peptide inhibitors, and antisense (RNAi) inhibitors.

Exemplary small molecule inhibitors include, without limitation, NSAIDs such as Indomethacin, Sulindac, Sulindac sulfone, Sulindac sulfide, Aspirin, Rofecoxib, Diclofenac, Celecoxib, Meloxicam, Etodolac, Nabumetone.

Exemplary small molecule inhibitors include quercetin (Park et al., “Quercetin, a potent inhibitor against beta-catenin/Tcf signaling in SW480 colon cancer cells,” Biochem Biophys Res Commun. 328(1):227-34 (2005), which is hereby incorporated by reference in its entirety); compounds such as ZTM000990, PKF118-310, PKF118-744, PKF115-584, PKF-222-815, CPG049090, PNU-74654, ICG-001, NS0668036, and others disclosed in Trosset et al., “Inhibition of protein-protein interactions: The discovery of druglike beta-catenin inhibitors by combining virtual and biophysical screening,” In Proteins: Structure, Function, and Bioinformatics 64(1):60-67 (2006) and Barker et al., “Mining the Wnt Pathway for Cancer Therapeutics,” Nature Reviews Drug Discovery 5:997-1014 (2006), each of which is hereby incorporated by reference in its entirety; LC-363 (Avalon Pharmaceuticals, Germantown, Md.); compounds such as N′-[(E)-(5-methyl-2-furyl)methylidene]-2-phenoxybenzohydrazide, N′-[(E)-1-(5-methyl-2-thienyl)ethylidene]-2-phenoxyacetohydrazide, 5-[2-(5-methyl-2-furyl)ethyl]-2-(2-thienyl)-1H-indole, 2-(2-furyl)-5-[(E)-2-(5-methyl-2-furyl)ethenyl]-1H-indole, N-[(E)-(5-methyl-2-furyl)methylidene]-4-(4-pyridinyl)-8-quinolin-amine, 2-(2-furyl)-5-[2-(5-methyl-2-furyl)ethyl]-1H-indole, 7-{(2E)-2-[(5-methyl-2-furyl)methylene]hydrazino}-N-(2-phenyleth-yl)-5,6-dihydrobenzo[h]isoquinoline-9-carboxamide, 1-{[(E)-(5-methyl-2-furyl)methylidene]amino}-3-(4-pyridinyl)-2,4-(1H,3H)-quinazolinedione, N-(5-methyl-2-furyl)-N-(2′-phenoxy[1,1′-biphenyl]-3-yl)amine, 4-{[7-(5-methyl-2-furyl)-2-naphthyl]oxy}pyridine, N-(5-bromo-1,3,4-oxadiazol-2-yl)-4-hydroxy-2-oxo-6-phenyl-2H-pyran-3-carboxamide, 4-hydroxy-N-(5-methyl-2-furyl)-2-oxo-6-phenyl-2H-pyran-3-carbox-amide, 3-[(E)-2-(5-bromo-1,3,4-thiadiazol-2-yl)ethenyl]-4-hydroxy-6-ph-enyl-2H-pyran-2-one, N-(5-bromo-1,3,4thiadiazol-2-yl)-4-hydroxy-2-oxo-6-phenyl-2H-pyran-3-carboxamide, 5-[(3-amino-1H-1,2,4-triazol-5-yl)methyl]-3-[3-fluoro-4-(4-morpholinyl)phenyl]-1,3-oxazolidin-2-one, 4-[(3-amino-1H-1,2,4-triazol-5-yl)methyl]-1-[3-fluoro-4-(4-morpholinyl)phenyl]-2-imidazolidinone, 1-benzhydryl-4-(5-bromo-2-furoyl)piperazine, 1-benzhydryl-4-[(5-methyl-2-thienyl)carbonyl]piperazine, benzyl (2E)-2-[1-(4-methyl-2-thienyl)ethylidene]hydrazinecarboxylate, 2-(4-chlorophenyl)-6-methyl-5-(5-methyl-1,3,4-oxadiazol-2-yl)[1-,3]thiazolo[3,2-b][1,2,4]triazole, N-(5-methyl-3-isoxazolyl)-N′-[(5-phenyl-1,3,4-oxadiazol-2-yl)carbonyl]urea, N-[3-(2-{[(5-chloro-2-thienyl)methyl]sulfonyl}hydrazino)-3-oxopropyl]benzenesulfonamide-5-[3-(4-phenoxyphenyl)propyl]-1,3,4-oxadiazol-2-ol, N-(3-methyl-5-isoxazolyl)-4-phenoxybenzamide, 4-hydroxy-N-(3-methyl-5-isoxazolyl)-2-oxo-6-phenoxy-2H-pyran-3-carboxamide, 2-phenoxy-N′-[(Z)-phenyl(2-thienyl)methylidene]benzo-hydrazide, 2-anilino-N′-[(Z)-2-furyl(phenyl)methylidene]benzohydrazide, 4-[(Z)-1-(3-methyl-5-isoxazolyl)-2-phenylethenyl]phenyl 2-(1-pyrrolidinyl)ethyl ether, 5-methyl-2-furaldehyde[(3Z)-2-oxo-1-(4-pyridinyl)-1,2-dihydro-3H-indol-3-ylidene]hydrazone, (2Z)—N-[(5-methyl-2-furyl)methyl]-2-[2-oxo-1-(4-pyridinyl)-1,2-dihydro-3H-indol-3-ylidene]ethanamide, (2Z)—N-[(3-methyl-5-isoxazolyl)methyl]-2-[2-oxo-1-(4-pyridinyl)-1,2-dihydro-3H-indol-3-ylidene]ethanamide, (2-chloro-1,3-thiazol-5-yl)methyl 4-(4-morpholinylsulfonyl)phenyl ether, N-(4,5-dihydronaphtho[1,2-d][1,3]thiazol-2-yl)-N-(4-phenoxybutyl)methanesulfonamide, N-(6-methoxy-4,5-dihydronaphtho[1,2-d][1,3]thiazol-2-yl)-N-[2-(1-methyl-3-phenylpropoxy)ethyl]acetamide, 4-{2-[(5-methyl-2-furyl)methoxy]benzylidene}-1-(4-pyridinylsulfonyl)piperidine, 4-{2-[(5-bromo-2-furyl)methoxy]benzylidene}-1-isonicotinoylpiperidine, N-(4,5-dihydronaphtho[1,2-d][1,3]thiazol-2-yl)-N-(4-phenylpentyl)acetamide, N-(4,5-dihydro-3H-naphtho[1,2-d]imidazol-2-yl)-N-[2-(2-phenylethoxy)ethyl]methanesulfonamide, N′-[(Z)-(5-methyl-2-furyl)(2-pyridinyl)methylidene]-2-phenoxybenzohydrazide, and their pharmaceutically acceptable salts as disclosed in US Patent Application Publ. No. 20040204477 to Moll et al., which is hereby incorporated by reference in its entirety; hydroxymatairesinol (U.S. Pat. No. 6,271,257 to Mutanen, which is hereby incorporated by reference in its entirety); hexachlorophene (Park et al., “Hexachlorophene Inhibits Wnt/[beta]-Catenin Pathway by Promoting Siah-Mediated [beta]-Catenin Degradation,” Molecular Pharmacology Fast Forward (May 30, 2006), which is hereby incorporated by reference in its entirety); and PPAR[gamma] agonists (e.g., troglitazone) and PPAR[gamma]-inactive analogs (e.g., [Delta]2TG and STG28) (Wei et al., “Thiazolidinediones Modulate the Expression of [beta]-Catenin and Other Cell-Cycle Regulatory Proteins by Targeting the F-Box Proteins of Skp1-Cull-F-box Protein E3 Ubiquitin Ligase Independently of Peroxisome Proliferator-Activated Receptor [gamma],” Molecular Pharmacology Fast Forward (Jun. 14, 2007), which is hereby incorporated by reference in its entirety).

Exemplary small molecule inhibitors include phytochemicals silibinin, milk thistle extract (cardio mariano), EGCG (epigallocatechin-3-gallate), White tea/Green tea, Sulforaphane, Resveratrol, Curcumin, Indole-3-carbinol, Ursolic acid, Docosahexanoic acid, Genistein, β-Lapachone.

Exemplary protein and peptide inhibitors include, without limitation, chibby overexpression (Schuierer et al., “Reduced expression of beta-catenin inhibitor Chibby in colon carcinoma cell lines,” World J Gastroenterol 12(10):1529-1535 (2006), which is hereby incorporated by reference in its entirety); Axin overexpression (Nakamura et al., “Axin, an inhibitor of the Wnt signalling pathway, interacts with beta-catenin, GSK-3beta and APC and reduces the beta-catenin level,” Genes Cells 3:395-403 (1998), which is hereby incorporated by reference in its entirety); HDPR1 overexpression (Yao et al., “HDPR1, a novel inhibitor of the WNT/beta-catenin signaling, is frequently downregulated in hepatocellular carcinoma: involvement of methylation-mediated gene silencing,” Oncogene 24:1607-1614 (2005), which is hereby incorporated by reference in its entirety); ICAT overexpression (Tago et al., “Inhibition of Wnt signaling by ICAT, a novel beta-catenin-interacting protein,” Genes Dev. 14:1741-1749 (2000); Genbank Accession No. BAB03458, each of which is hereby incorporated by reference in its entirety); and LXXLL (SEQ ID NO: 19) peptides of the type disclosed in U.S. Pat. No. 6,677,116 to Blaschuk et al., which is hereby incorporated by reference in its entirety. These protein or polypeptide inhibitors can be administered directly or expressed in vivo via gene therapy approaches, discussed below.

Exemplary antisense beta-catenin constructs include those reported in Green et al., “Beta-catenin Antisense Treatment Decreases Beta-catenin Expression and Tumor Growth Rate in Colon Carcinoma Xenografts,” J Surg. Res. 101(1):16-20 (2001); Veeramachaneni, “Down-regulation of Beta Catenin Inhibits the Growth of Esophageal Carcinoma Cells,” J. Thoracic Cardiovasc. Surg. 127(1):92-98 (2004); U.S. Pat. No. 6,066,500 to Bennett et al., each of which is hereby incorporated by reference in its entirety.

Exemplary siRNA constructs are described in Verma et al., “Small Interfering RNAs Directed Against Beta-catenin Inhibit the in vitro and in vivo Growth of Colon Cancer Cells,” Clin. Cancer Res. 9(4):1291-300 (2003), which is hereby incorporated by reference in its entirety; and other siRNA against beta-catenin are commercially available from Santa Cruz Biotechnology, Inc.

Exemplary shRNA constructs are described in Gadue et al., “Wnt and TGF-[beta] Signaling are Required for the Induction of an in vitro Model of Primitive Streak Formation using Embryonic Stem Cells,” Proc. Natl. Acad. Sci. USA 103(45):16806-16811, which is hereby incorporated by reference in its entirety; and other shRNA against beta-catenin are commercially available from Super Array Bioscience Corporation, OriGene, and Open Biosystems.

The RNAi agents can be administered directly or administered via gene therapy approach. Thus, DNA molecules (expression vectors) encoding these RNAi agents can also be administered.

For gene therapy approaches, the therapeutic agent, whether a polypeptide or an RNA molecule, can be administered to a patient in the form of a DNA molecule that expresses the therapeutic agent. In vivo, following administration of the DNA molecule, the therapeutic agent is expressed and can exert its effect on the patient for treating and/or preventing a pathology characterized by vascular malformation.

Nucleic acid agents (including RNA and DNA) for use in the methods of the present invention can be delivered to a subject in a number of ways known in the art, including through the use of gene therapy vectors and methods as described above. The nucleic acid can be contained within a vector useful for gene therapy, for example, a vector that can be transferred to the cells of a subject and provide for expression of the therapeutic nucleic acid agent therein. Such vectors include chromosomal vectors (e.g., artificial chromosomes), non-chromosomal vectors, and synthetic nucleic acids. Vectors also include plasmids, viruses, and phages, such as retroviral vectors, lentiviral vectors, adenoviral vectors, and adeno-associated vectors.

Nucleic acid agents can be transferred into a subject using ex vivo or in vivo methods. Ex vivo methods involve transfer of the nucleic acid into cells in vitro (e.g., by transfection, infection, or injection) that are then transferred into or administered to the subject. The cells can be, for example, cells derived from the subject (e.g., lymphocytes) or allogeneic cells. For example, the cells can be implanted directly into a specific tissue of the subject or implanted after encapsulation within an artificial polymer matrix. Nucleic acids can also be delivered into a subject in vivo. For example, nucleic acids can be administered in an effective carrier, e.g., any formulation or composition capable of effectively delivering the nucleic acid to cells in vivo. Nucleic acids contained within viral vectors can be delivered to cells in vivo by infection or transduction using virus. Nucleic acids and vectors can also be delivered to cells by physical means, e.g., by electroporation, lipids, cationic lipids, liposomes, DNA gun, calcium phosphate precipitation, injection, or delivery of naked nucleic acid.

As an alternative to non-infective delivery of nucleic acids as described above, naked DNA or infective transformation vectors can be used for delivery, whereby the naked DNA or infective transformation vector contains a recombinant gene that encodes, for example, the polypeptide or nucleic acid inhibitor of beta-catenin. The nucleic acid molecule is then expressed in the transformed cell.

The recombinant gene includes, operatively coupled to one another, an upstream promoter operable in mammalian cells and optionally other suitable regulatory elements (i.e., enhancer or inducer elements), a coding sequence that encodes the therapeutic nucleic acid (described above) or polypeptide, and a downstream transcription termination region. Any suitable constitutive promoter or inducible promoter can be used to regulate transcription of the recombinant gene, and one of skill in the art can readily select and utilize such promoters, whether now known or hereafter developed. The promoter can also be specific for expression in the vascular endothelium such as Tie-2 promoter or VE-cadherin promoter (Corada Metal. Nature Comm 4:2609, 2013); other brain endothelium specific promoters can also be used such as Slco1c1 (D. A. Ridder et al. J Exp Med 208 (13):2615, 2011).

Tissue specific promoters can also be made inducible/repressible using, e.g., a Tet0 response element. Other inducible elements can also be used. Known recombinant techniques can be utilized to prepare the recombinant gene, transfer it into the expression vector (if used), and administer the vector or naked DNA to a patient. Exemplary procedures are described in Sambrook et al., 1-3 MOLECULAR CLONING: A LABORATORY MANUAL (2d ed. 1989), which is hereby incorporated by reference in its entirety. One of skill in the art can readily modify these procedures, as desired, using known variations of the procedures described therein.

Any suitable viral or infective transformation vector can be used. Exemplary viral vectors include, without limitation, adenovirus, adeno-associated virus, and retroviral vectors (including lentiviral vectors).

It is preferable that the therapeutic agent is administered to the patient in the form of a pharmaceutical composition that includes a pharmaceutically acceptable carrier and one or more active agents that inhibit beta-catenin activity directly by acting on beta-catenin, by promoting beta-catenin degradation, indirectly competing with other agents for binding with beta-catenin, or by interfering with the expression of beta-catenin.

The pharmaceutical compositions of the present invention are preferably in the form of a single unit dosage form that contains an amount of the therapeutic agent that is effective to treat and/or prevent a pathology characterized by vascular malformation of the type described herein. The pharmaceutical composition can also include suitable excipients, or stabilizers, and can be in solid or liquid form such as, tablets, capsules, powders, solutions, suspensions, or emulsions. Typically, the composition will contain from about 0.01 to 99 percent, preferably from about 5 to 95 percent of active compound(s), together with the carrier. The therapeutic agent, when combined with a suitable carrier and any excipients or stabilizers, and whether administered alone or in the form of a composition, can be administered orally, parenterally, subcutaneously, transdermally, intravenously, intramuscularly, intraperitoneally, by intranasal instillation, by implantation, by intracavitary or intravesical instillation, intraocularly, intraarterially, intralesionally, by application to mucous membranes, such as, that of the nose, throat, and bronchial tubes (i.e., inhalation), or by intracerebral administration.

For most therapeutic purposes, the therapeutic can be administered orally as a solid or as a solution or suspension in liquid form, via injection as a solution or suspension in liquid form, or via inhalation of a nebulized solution or suspension.

The solid unit dosage forms containing the therapeutic agent can be of a conventional type. The solid form can be a capsule, such as an ordinary gelatin type containing the therapeutic agent and a carrier, for example, lubricants and inert fillers such as, lactose, sucrose, or cornstarch. In another embodiment, the therapeutic agent is tableted with conventional tablet bases such as lactose, sucrose, or cornstarch in combination with binders like acacia or gelatin, disintegrating agents such as cornstarch, potato starch, or alginic acid, and a lubricant such as stearic acid or magnesium stearate.

For injectable dosages, solutions or suspensions of the therapeutic agent can be prepared in a physiologically and pharmaceutically acceptable diluent as the carrier. Such carriers include sterile liquids, such as water and oils, with or without the addition of a surfactant and other pharmaceutically and physiologically acceptable components, including adjuvants, excipients or stabilizers. Illustrative oils are those of petroleum, animal, vegetable, or synthetic origin, for example, peanut oil, soybean oil, or mineral oil. In general, water, saline, aqueous dextrose and related sugar solutions, and glycols, such as propylene glycol or polyethylene glycol, are preferred liquid carriers, particularly for injectable solutions.

For use as aerosols, the therapeutic agent in solution or suspension may be packaged in a pressurized aerosol container together with suitable propellants, for example, hydrocarbon propellants like propane, butane, or isobutane with conventional adjuvants. The therapeutic agent also may be administered in a non-pressurized form such as in a nebulizer or atomizer.

In addition to the above-described formulations which are intended to immediately deliver the therapeutic agents to the patient, sustained release formulations are also contemplated. Preferably, the sustained release formulation is an implantable device that includes a matrix in which the therapeutic agent is captured. Release of the agents can be controlled via selection of materials and the amount of drug loaded into the vehicle. A number of suitable implantable delivery systems are known in the art, such as U.S. Pat. No. 6,464,687 to Ishikawa et al., U.S. Pat. No. 6,074,673 to Guillen, each of which is hereby incorporated by reference in its entirety.

Implantable, sustained release drug delivery systems can be formulated using any suitable biocompatible matrix into which an agent can be loaded for sustained-release delivery. These include, without limitation, microspheres, hydrogels, polymeric reservoirs, cholesterol matrixes, polymeric systems and non-polymeric systems, etc. Exemplary polymeric matrixes include, without limitation, poly(ethylene-co-vinyl acetate), poly-L-lactide, poly-D-lactide, polyglycolide, poly(lactide-co-glycolide), polyanhydride, polyorthoester, polycaprolactone, polyphospagene, proteinaceous polymer, polyether, silicone, and combinations thereof.

Alternatively, for DNA-based therapeutic agents, one suitable vehicle for delivering the therapeutic agent includes solubilized cholesterol as an additive for DNA complexed with a cationic lipid, a cationic polymer, or a dendrimer. Preferably, the cholesterol is solubilized using a cyclodextrin, preferably methyl[beta]-cyclodextrin. This type of formulation is described in U.S. Patent Publ No. 20020146830 to Esuvaranathan et al., which is hereby incorporated by reference in its entirety.

Use of the inhibitor of Wnt/beta-catenin signaling in combination with one or more other therapeutic agents is also contemplated. For example, for the treatment of a pathology characterized by vascular malformation, treatment with one of the above-identified inhibitors of Wnt/beta-catenin signaling in combination with another known treatment of a pathology characterized by vascular malformation including, without limitation, anti-oxidant, TGF-β signaling pathway inhibitors, BMP signaling pathway inhibitors, VEGF signaling pathway inhibitors, Yap signaling pathway inhibitors, statins (see for example Hwang et al, 2013, Int J. Oncol 43, 261-270) and other inhibitors of RhoA GTPase levels and/or activity and combinations thereof.

Thus, the present invention also relates to formulations and therapeutic systems comprising two or more active agents, one of which is the inhibitor of beta-catenin. Preferred inhibitors of the invention are further listed below selected from:

Sulindac, also known as (Z)-5-Fluoro-2-methyl-1-[4-(methylsulfinyl)benzylidene]indene-3-acetic acid or 2-[(3Z)-6-fluoro-2-methyl-3-[(4-methylsulfinylphenyl)methylene]inden-1-yl]acetic acid; sulindac sulphide, also known as (Z)-5-Fluoro-2-methyl-1-[4-(methylthio)benzylidene]indene-3-acetic acid or 2-[(3Z)-6-fluoro-2-methyl-3-[(4-methylsulfanylphenyl)methylene]inden-1-yl]acetic acid; sulindac sulfone, also known as (Z)-5-Fluoro-2-methyl-1-[4-(methylsulfonyl)benzylidene]indene-3-acetic acid or 2-[(3Z)-6-fluoro-2-methyl-3-[(4-methylsulfonylphenyl)methylene]inden-1-yl]acetic acid; phospho-sulindac, also known as 4-diethoxyphosphoryloxybutyl-(Z)-5-Fluoro-2-methyl-1-[4-(methylsulfinyl)benzylidene]indene-3-acetate or 4-diethoxyphosphoryloxybutyl 2-[(3Z)-6-fluoro-2-methyl-3-[(4-methylsulfinylphenyl)methylene]inden-1-yl]acetate; phospho-sulindac sulphide, also known as 2-[(3Z)-6-fluoro-2-methyl-3-[(4-methylsulfanylphenyl)methylene]inden-1-yl]acetate: phospho-sulindac sulfone, known as 4-diethoxyphosphoryloxybutyl 2-[(3Z)-6-fluoro-2-methyl-3-[(4-methylsulfonylphenyl)methylene]inden-1-yl]acetate; silibinin, also known as 2,3-Dihydro-3-(4-hydroxy-3-methoxyphenyl)-2-(hydroxymethyl)-6-(3,5,7-trihydroxy-4-oxobenzopyran-2-yl)benzodioxin; curcumin, also known as (E,E)-1,7-bis(4-Hydroxy-3-methoxyphenyl)-1,6-heptadiene-3,5-dione; resveratrol, also known as 3,4′,5-trihydroxy-trans-stilbene; salinomycin, and the pharmaceutically acceptable salts thereof and analogs thereof. Analogs are compounds similar in structure but different in respect to elemental composition. Structural research is very active for producing analogs, in particular of sulindac metabolites, to identify chemical modifications (e.g., phosphorylation⁵⁷ or benzylamide derivatization, Whitt et al, 2012, Cancer Prev. Res. 5, 822-833) that might enhance their activity while reducing their toxicity.

“Pharmaceutically acceptable salts” comprise conventional non-toxic salts obtained by salification with organic or inorganic bases. The inorganic salts are, for example, metal salts, particularly alkali metal salts, alkaline-earth metal salts and transition metal salts (such as sodium, potassium, calcium, magnesium, aluminum). Salts may be also obtained with bases, such as ammonia or secondary or tertiary amines (such as diethylamine, triethylamine, piperidine, piperazine, morpholine), or with basic amino-acids, or with osamines (such as meglumine), or with aminoalcohols (such as 3-aminobutanol and 2-aminoethanol).

In addition, the compounds of the present invention can exist in unsolvated as well as in solvated forms with pharmaceutically acceptable solvents such as water, ethanol and the like.

The invention also comprises pharmaceutical compositions characterized by containing one or more active principles selected from sulindac, sulindac sulfide, sulindac sulfone, phospho-sulindac, phospho-sulindac sulphide, phospho-sulindac sulfone, silibinin, curcumin, resveratrol, and salinomycin, in association with pharmaceutically acceptable carrier, excipients and diluents for the use in the treatment of a pathology characterized by vascular malformation, in particular Cerebral Cavernous Malformation (CCM).

The compounds of this invention can be administered via any of the accepted modes of administration or agents for serving similar utilities. Thus, administration can be, for example, oral, nasal, parental (intravenous, subcutaneous, intramuscular), buccal, sublingual, rectal, topical, transdermal, intravesical, or using any other route of administration.

The compounds can be pharmaceutically formulated according to known methods. The pharmaceutical compositions can be chosen on the basis of the treatment requirements. Such compositions are prepared by blending and are suitably adapted to oral or parenteral administration, and as such can be administered in the form of tablets, capsules, oral preparations, powders, granules, pills, injectable or infusible liquid solutions, suspensions or suppositories.

Tablets and capsules for oral administration are normally presented in unit dose form and contain conventional excipients such as binders, fillers, diluents, tableting agents, lubricants, detergents, disintegrants, coloring agents, flavoring agents and wetting agents. The tablets can be coated using methods well known in the art.

Suitable fillers include cellulose, mannitol, lactose and other similar agents. Suitable disintegrants include polyvinylpyrrolidone and starch derivatives such as sodium glycolate starch. Suitable lubricants include, for example, magnesium stearate. Suitable wetting agents include sodium lauryl sulfate.

The oral solid compositions can be prepared by conventional methods of blending, filling or tableting. The blending operation can be repeated to distribute the active principle throughout compositions containing large quantities of fillers. Such operations are conventional.

Oral liquid preparations can be in the form of, for example, aqueous or oily suspensions, solutions, emulsions, syrups or elixirs, or can be presented as a dry product for reconstitution with water or with a suitable vehicle before use. Such liquid preparations can contain conventional additives such as suspending agents, for example sorbitol, syrup, methyl cellulose, gelatin, hydroxyethyl cellulose, carboxymethyl cellulose, aluminium stearate gel, or hydrogenated edible fats; emulsifying agents, such as lecithin, sorbitan monooleate, or acacia; non-aqueous vehicles (which can include edible oils), such as almond oil, fractionated coconut oil, oily esters such as esters of glycerine, propylene glycol, or ethyl alcohol; preservatives, such as methyl or propyl p-hydroxybenzoate or sorbic acid, and if desired, conventional flavoring or coloring agents. Oral formulations also include conventional slow-release formulations such as enterically coated tablets or granules.

For parenteral administration (e.g. bolus injection or continuous infusion), fluid unit dosages (e.g. in ampoules or in multi-dose containers) can be prepared, containing the compound and a sterile vehicle. The compound can be either suspended or dissolved, depending on the vehicle and concentration. The parenteral solutions are normally prepared by dissolving the compound in a vehicle, sterilising by filtration, filling suitable vials and sealing. Advantageously, adjuvants such as local anaesthetics, preservatives and buffering agents can also be dissolved in the vehicle. To increase stability, the composition can be frozen after having filled the vials and removed the water under vacuum. Parenteral suspensions are prepared substantially in the same manner, except that the compound can be suspended in the vehicle instead of being dissolved, and sterilized by exposure to ethylene oxide before suspending in the sterile vehicle. Advantageously, a surfactant or wetting agent can be included in the composition to facilitate uniform distribution of the compound of the invention.

For buccal or sublingual administration the compositions may be tablets, lozenges, pastilles, or gel.

The compounds can be pharmaceutically formulated as suppositories or retention enemas, e.g. containing conventional suppositories bases such as cocoa butter, polyethylene glycol, or other glycerides, for a rectal administration.

Another means of administering the compounds of the invention regards topical treatment. Topical formulations can contain for example ointments, creams, lotions, gels, solutions, pastes and/or can contain liposomes, micelles and/or microspheres. Examples of ointments include oleaginous ointments such as vegetable oils, animal fats, semisolid hydrocarbons, emulsifiable ointments such as hydroxystearin sulfate, anhydrous lanolin, hydrophilic petrolatum, cetyl alcohol, glycerol monostearate, stearic acid, water soluble ointments containing polyethylene glycols of various molecular weights. Creams, as known to formulation experts, are viscous liquids or semisolid emulsions, and contain an oil phase, an emulsifier and an aqueous phase. The oil phase generally contains petrolatum and an alcohol such as cetyl or stearic alcohol. The emulsifier in a cream formulation is chosen from non-ionic, anionic, cationic or amphoteric surface-active agents. Dispersing agents such as alcohol or glycerin can be added for gel preparation. The gelling agent can be dispersed by finely chopping and/or mixing.

A further method of administering the compounds of the invention regards transdermal delivery. Typical transdermal formulations comprise conventional aqueous and non-aqueous vectors, such as creams, oils, lotions or pastes or can be in the form of membranes or medicated patches.

A reference for the formulations is the book by Remington (“Remington: The Science and Practice of Pharmacy”, Lippincott Williams & Wilkins, 2000).

Administration through synthetic nanoparticles engineered to target pathological endothelial cells, for example expressing EndMT markers as in CCM lesions (such as Klf4, Klf2, Ly6a, S100a4, CD44, Id1, a-Sma, Slug, PAI1, N-cadherin, Zeb2, other markers are indicated in Fadini et al, 2012: Margariti et al, 2012; Li et al, 2012; Liang et al 2011; Stein et al, 2006; Medici et al, 2012) is also comprised within the present invention (Davis et al, 2010, Nature 464, 1067-1071; Dashi et al, 2012 Adv Mater., 24, 3864-3869). Small molecules, proteins, peptide, antisense nucleic acid may be encapsulated in such nanoparticles.

The above mentioned uses and methods also include the possibility of co-administration of additional therapeutic agents, simultaneously or delayed with respect to the administration of the compounds selected from sulindac, sulindac sulfide, sulindac sulfone, phospho-sulindac, phospho-sulindac sulphide, phospho-sulindac sulfone, silibinin, curcumin (see for example Cheng et al, 2013, Int J. of Oncology 43, 895-902), resveratrol, and salinomycin.

In the previously mentioned uses and methods, the dosage of the compounds selected from sulindac, sulindac sulfide, sulindac sulfone, phospho-sulindac, phospho-sulindac sulphide, phospho-sulindac sulfone, silibinin, curcumin, resveratrol, and salinomycin, can vary depending upon a variety of factors including the patient type and condition, the degree of disease severity, mode and time of administration, diet and drug combinations. As an indication, they can be administered within a dose range of between 0.001 and 1000 mg/kg/day. The determination of optimum dosages for a particular patient is well known to one skilled in the art. Preferred dose range is between 1 and 10 mg/kg/day, most preferred range is between 10 and 100 mg/kg/day. Still preferred dose range is between 100 and 200 mg/kg/day. Yet preferred dose range is between 200 and 500 mg/kg/day. Still preferred dose range is between 500 and 1000 mg/kg/day. Preferably the inhibitor of the invention is administered orally.

As is common practice, the compositions are normally accompanied by written or printed instructions for use in the treatment in question.

The present invention will be illustrated by means of non-limiting examples referring to the following figures:

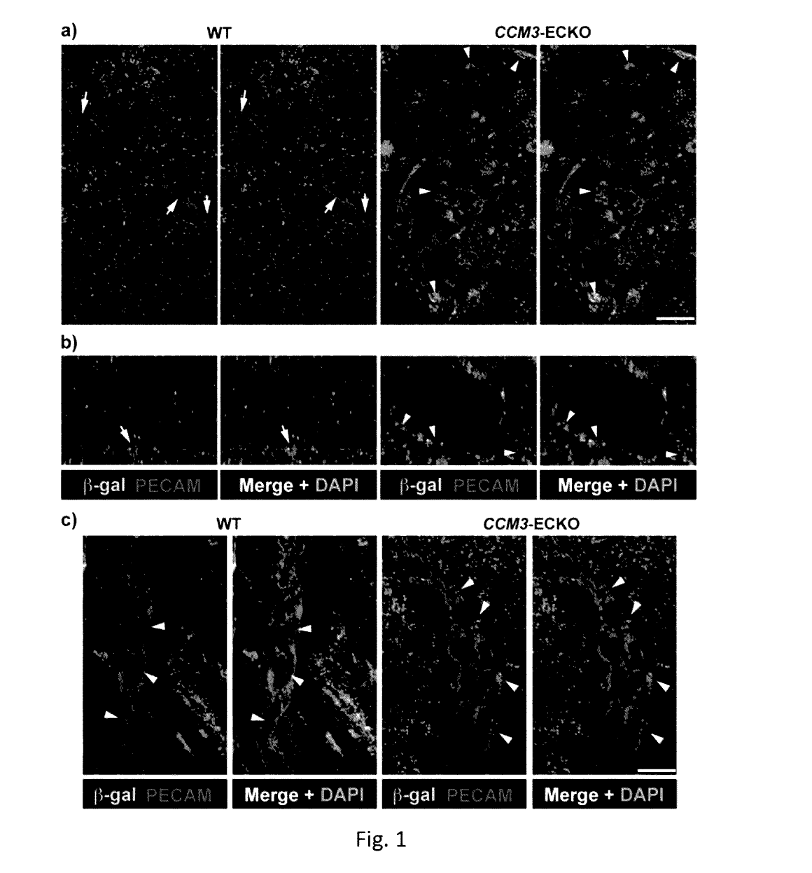

FIG. 1. Endothelial cells in brain and retina vessels of CCM3-ECKO (endothelial-cell-specific homozygous deletion of CCM3) mice show enhanced b-catenin transcription activity. a-c. Representative immunostaining of brain sections (a, b) and retinas (c, flat-mount) (a, c, XY axis; b, Z projection along X axis) from wild-type (WT) and CCM3-ECKO mice (mice with endothelial-specific-inactivation of CCM3 gene), for b-gal, as a gene reporter of b-catenin transcription activity in Pecam-positive (endothelial) cells. Nuclei were stained with DAPI. Arrows, b-gal-negative nuclei; arrowheads, b-gal-positive nuclei. Endothelial cell nuclei were counted in 50 random fields at 63× magnification. The quantification by random-field counting using Pecam labeling of endothelial cells (see Methods) saw the b-gal-positive nuclei (0.86±0.15 positive nuclei per field; positive nuclei were 7.2% of 600 total endothelial nuclei) of the control brain endothelial cells from the wild-type BAT-gal mice significantly increased by 6-fold (6.0±1.7 positive nuclei per field; positive nuclei were 42.9% of 700 total endothelial nuclei) in the CCM3-ECKO brain endothelial cells (p<0.05; t-test). In these CCM3-ECKO brain endothelial cells, the b-gal-positive nuclei were similarly distributed both in established caverns and in telangiectases.

b-Gal-positive nuclei were more abundant in both brain and retina endothelial cells from CCM3-ECKO mice than in control cells. For the retina (c), two veins in the middle area are shown. Samples in a-c are from dpn 9 littermate pups. Scale bars, 20 mm.

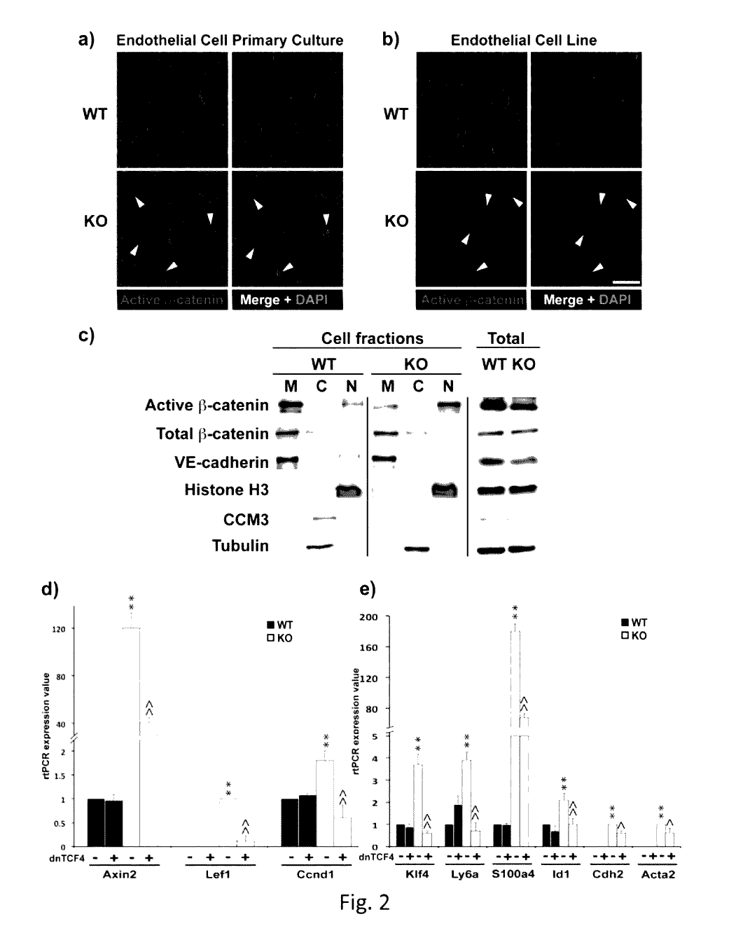

FIG. 2. CCM3-knockout endothelial cells in culture show delocalization of active b-catenin from cell-cell junctions and its concentration into the nucleus, where it is transcriptionally active, a, b. Representative immunostaining of wild-type (WT) and CCM3-knockout (KO) endothelial cells for active b-catenin. Primary culture (a) and cell line (b). Nuclei were stained with DAPI. Arrowheads, b-catenin-positive nuclei. In these CCM3-knockout endothelial cells, the CCM3 transcript was reduced by 70% to 90% (primary culture) and not detectable (line) by rtPCR. Scale bar, 20 mm. c. Representative cell fractionation and Western blotting of membrane (M), cytoplasm (C) and nucleus (N) compartments of wild-type (WT) and CCM3-knockout (KO) endothelial cell line. Total, membrane and nucleus active b-catenin were decreased by 34% to 42%, decreased by 58% to 75%, and increased by 51% to 66%, respectively; active b-catenin was almost undetectable in the cytoplasm in CCM3-knockout versus WT endothelial cells. In wild-type, CCM3 was enriched in the cytoplasm; in the CCM3 knockout, no CCM3 was detected by Western blotting. d, e. Quantification of typical b-catenin transcription targets (d) and of endothelial progenitor phenotype/EndMT markers (e) without (−) and with (+) expression of dominant-negative Tcf4, in wild-type (WT) and CCM3-knockout (KO) endothelial cell line. Data are means (±SD) of triplicate rtPCR assays from three independent experiments. Tubulin transcripts a and b, which are not targets of CCM3 knockout, were not modified by dominant-negative Tcf4 (unpublished result). **, p<0.01 for CCM3 knockout versus control (WT). ̂, p<0.05; ̂̂, p<0.01 for CCM3 knockout plus dominant-negative Tcf4 (+) versus CCM3 knockout plus GFP (−) (t-test).

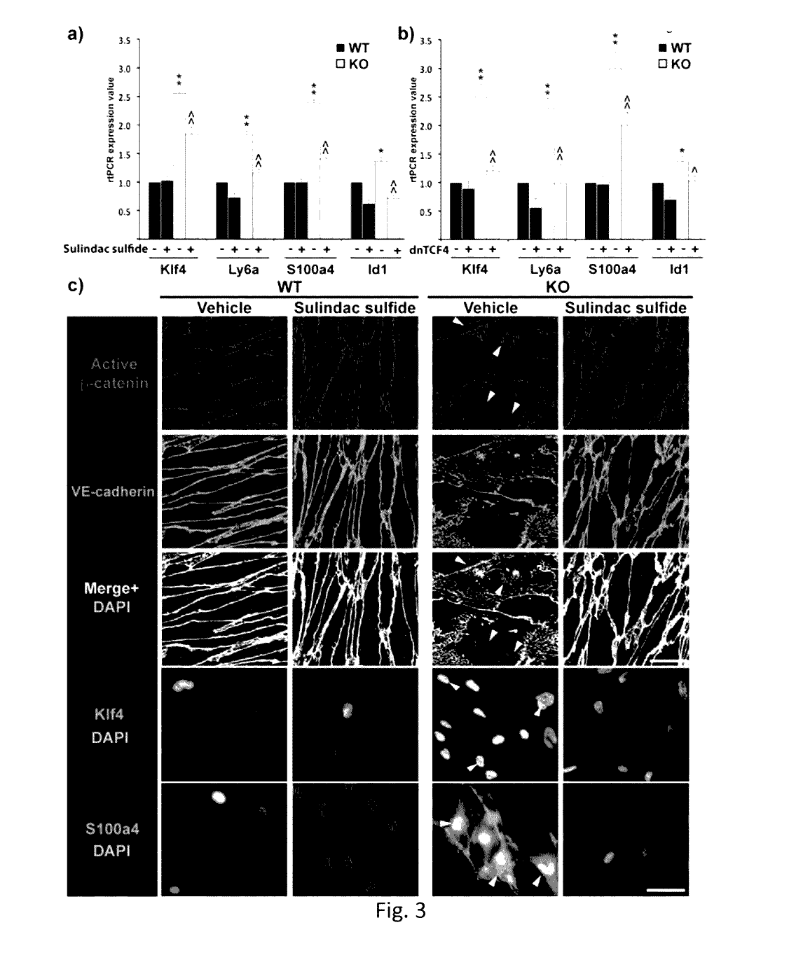

FIG. 3. CCM3-knockout endothelial cells show sulindac sulfide inhibition of transcription of b-catenin target genes and induction of re-localization of active b-catenin from the nucleus to adherens junctions. a. Quantification of sulindac sulfide inhibition of transcription of b-catenin target genes (see b) Klf4, Ly6a, S100a4 and Id1 by rtPCR, in wild-type (WT) and CCM3-knockout (KO) brain endothelial cells in primary culture. *, p<0.05; **, p<0.01 (t-test) for the comparison CCM3-knockout versus WT under basal conditions. ̂̂, p<0.01 (t-test) for the comparison CCM3 knockout plus sulindac sulfide versus vehicle treated-CCM3 knockout. b. Quantification of dominant-negative Tcf4 inhibition of transcription of genes tested in (a) by rtPCR. *, p<0.05; **, p<0.01 (t-test) for the comparison CCM3-knockout versus WT under basal conditions. ̂, p<0.05; ̂̂, p<0.01 (t-test) for the comparison CCM3 knockout plus dominant-negative Tcf4 (+) sulfide versus CCM3 knockout plus GFP (−). c. Representative immunostaining of brain endothelial cells in primary culture under sulindac sulfide treatment. Sulindac-sulfide-mediated redistribution of active b-catenin (arrowheads in vehicle-treated KO) from the nucleus (see corresponding arrowheads in DAPI staining) to cell-cell junctions in these CCM3-knockout endothelial cells. Co-localization of active b-catenin and VE-cadherin is observed after sulindac sulfide treatment in KO. Scale bar, 15 mm. Bottom two rows: Sulindac sulfide inhibition of overexpression of Klf4 and S100a4 (white nuclei arrowheads) in these CCM3-knockout endothelial cells. Total nuclei are DAPI-positive or outlined by white lines. Klf4 is exclusively nuclear and S100a4 is both nuclear and cytoplasmic in these CCM3-knockout endothelial cells. Scale bar, 30 mm.

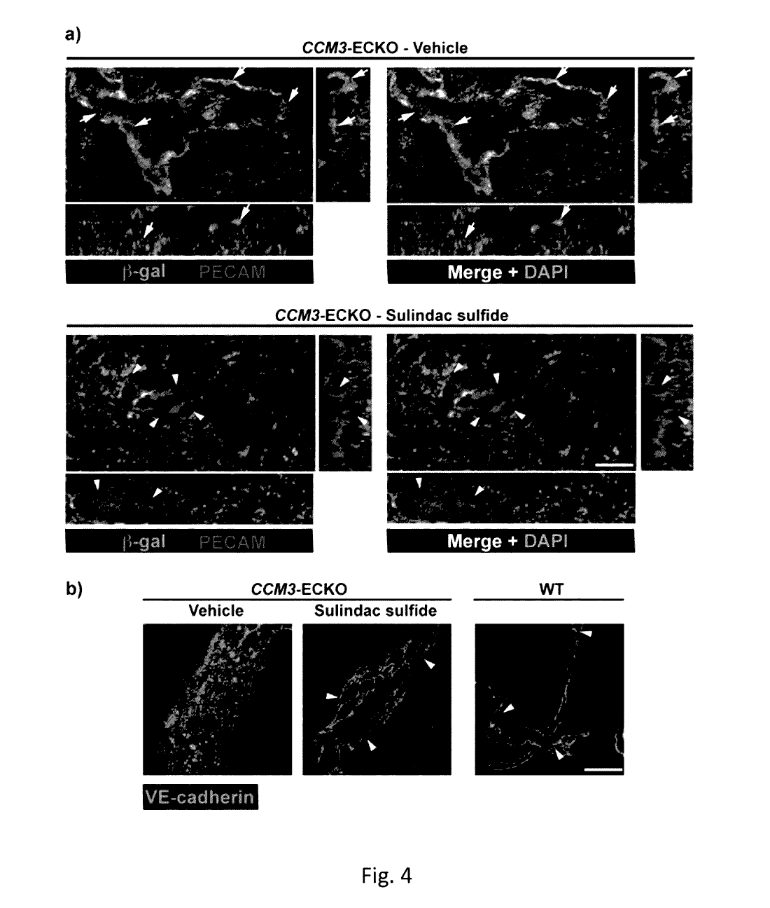

FIG. 4. Endothelial cells in brain vessels of CCM3-ECKO mice show sulindac sulfide inhibition of b-catenin transcription activity and induction of re-localization of VE-cadherin from diffused distribution to adherens junctions. Representative immunostaining of brain sections without (vehicle) and with sulindac sulfide treatment of the CCM3-ECKO mice. a. Sulindac-sulfide-mediated abolition of b-gal reactivity in the nucleus (top, arrows, versus bottom, arrowheads), as a gene reporter of b-catenin transcription activity in Pecam-positive (endothelial) cells. Each panel shows XY axis (main image), and Z projection along X axis (below). Nuclei were stained with DAPI. Scale bar, 25 mm. b. Sulindac-sulfide-mediated redistribution of VE-cadherin from diffused distribution to cell-cell junctions (middle panel, arrowheads) in these blood-vessel CCM3-ECKO endothelial cells, for a distribution similar to matched wild-type (WT) mice (right panel, arrowheads). Sections in a and b are from dpn 9 littermate pups. Scale bar, 30 mm.

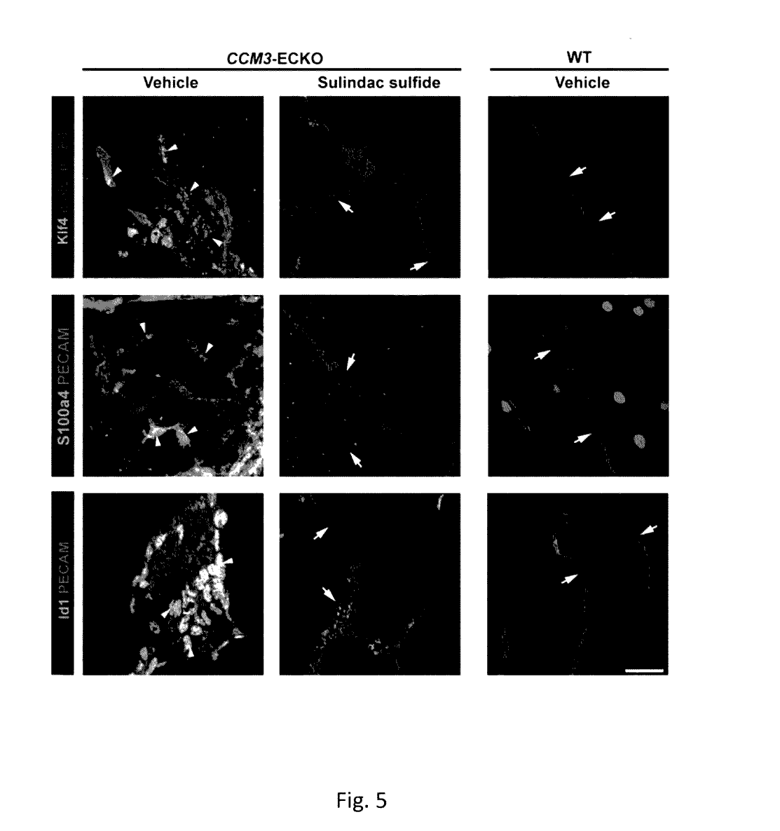

FIG. 5. Endothelial cells in brain vessels of CCM3-ECKO mice show sulindac sulfide inhibition of overexpression of progenitor and EndMT markers.

Representative immunostaining shows Klf4 (top, arrowheads) S100a4 (middle, arrowheads), and Id1 (bottom, arrowheads) concentrated in the nuclei of these blood-vessel CCM3-ECKO endothelial cells (Pecam positive; isolectin B4 positive). Sulindac sulfide strongly reduces this nuclear reactivity for Klf4, S100a4 and Id1 (arrows), for a distribution similar to matched wild-type (WT) mice (right panels, arrows). Brain sections are from dpn 9 littermate pups. Scale bar, 30 mm.

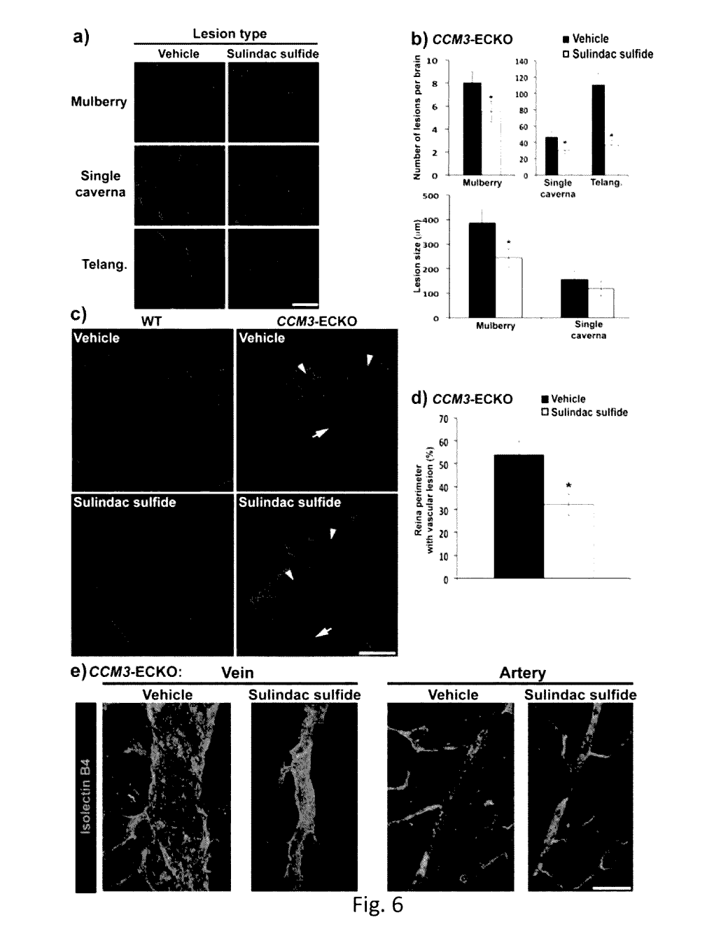

FIG. 6. Brain and retina vessels in CCM3-ECKO mice show sulindac-sulfide-induced reduction of lesions. a. Representative immunostaining of vascular lesions of brain sections without (vehicle) and with sulindac sulfide treatment of CCM3-ECKO mice, as mulberry (multiple cavernae), single caverna and telangiectases (Telang.). Lesions are classified as in⁶³. b. Top panels: quantification of brain lesions as illustrated in (a) (see methods for details). Littermates (dpn 9 pups) from five independent litters: vehicle treated (n=8) or sulindac sulfide treated (n=7). *, p<0.005, Wilcoxon signed-rank test. Bottom panel: quantification of brain lesion sizes (mm, see Methods). *, p<0.05, t-test. c. Representative immunostaining for Pecam (endothelial cells) of vessels in the retina of wild-type (WT) and CCM3-ECKO mice (dpn 9 littermate pups) treated without (vehicle) and with sulindac sulfide. Multiple-lumen vascular lesions (arrowheads) develop from veins (arrow). Sulindac sulfide (bottom right) reduces the malformations (arrowheads) and vein diameter (arrow), (see also e). d. Quantification of the retina vascular lesions illustrated in (c) as percentages of retinal perimeter affected by vascular lesions (n=14 for both vehicle and sulindac sulfide, see methods). *, p<0.05, t-test. e. Representative immunostaining of the retina vascular lesions illustrated in (c). As well as the peripheral vascular malformations, sulindac sulfide induced reductions in vein diameters (see text for details). Arteries of these CCM3-ECKO mice do not show this aberrant phenotype (endothelial cells isolectin B4-labelling) Scale bars, 100 mm (a); 700 mm (c); 60 mm (e).

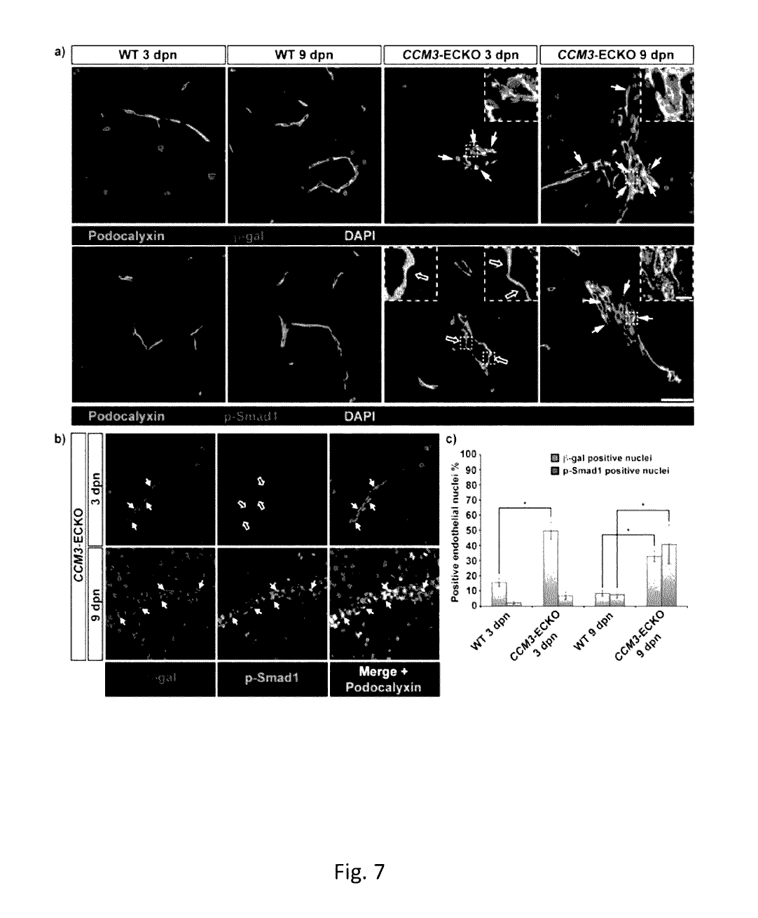

FIG. 7. Brain endothelial cells in CCM3-ECKO mice show enhanced β-catenin transcription activity earlier than activation of TGF-8/BMP signaling. a. Representative immunostaining of brain sections from wild-type (WT) and CCM3-ECKO mice, for β-gal (red, upper panel), as a gene reporter of β-catenin transcription activity and p-Smad1 (red, lower panel), as a marker of activation of TGF-β/BMP signaling, in endothelial cells (Podocalyxin-positive, green) at early (3dpn) and late (9dpn) time points after CCM3 recombination (1dpn). Nuclei, DAPI-stained, are blue. b. Co-staining for β-gal (red), p-Smad1 (green) and Podocalyxin (blue) is shown. In (a) and (b), arrows, β-gal- and p-Smad1-positive nuclei; empty arrows, p-Smad1 negative nuclei in endothelial cells of a vascular malformations (caverna in a) and telangiectasia in b) in 3dpn pups. Insets, magnification of boxed areas. Scale bars, 50 μm; inset in a) 10 μm. c. Quantification of β-gal-positive and p-Smad1 positive endothelial cells in brain sections of WT and CCM3-ECKO pups at 3 and 9dpn. A total number of at least 450 nuclei were counted in forty random fields at 63× magnification for each condition in samples from matched littermate pups in three independent experiments. *, p<0.01 versus indicated controls (t-test).

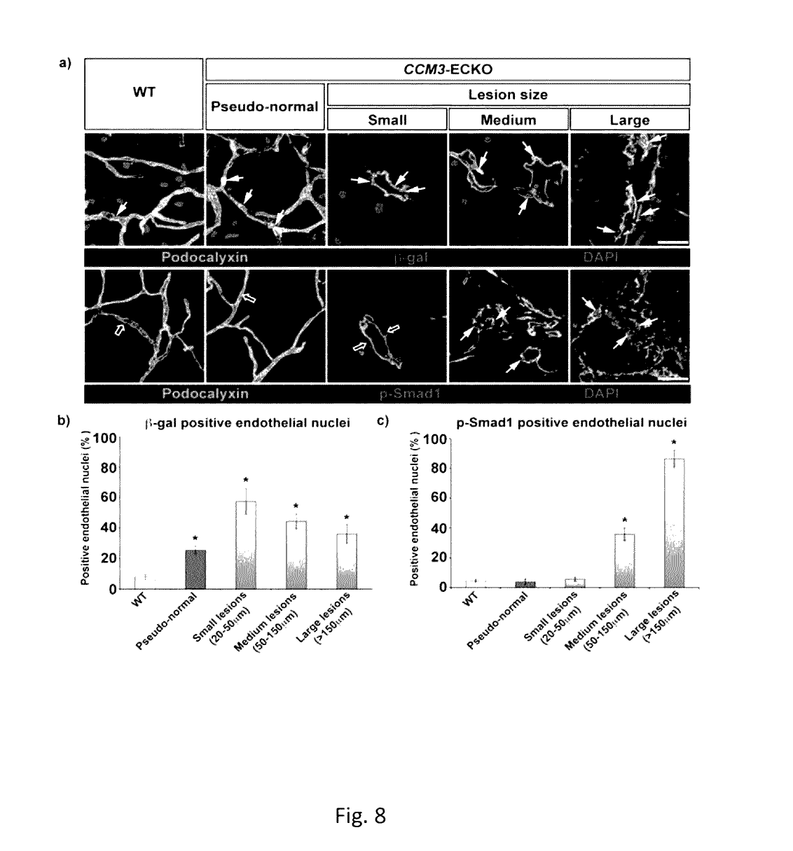

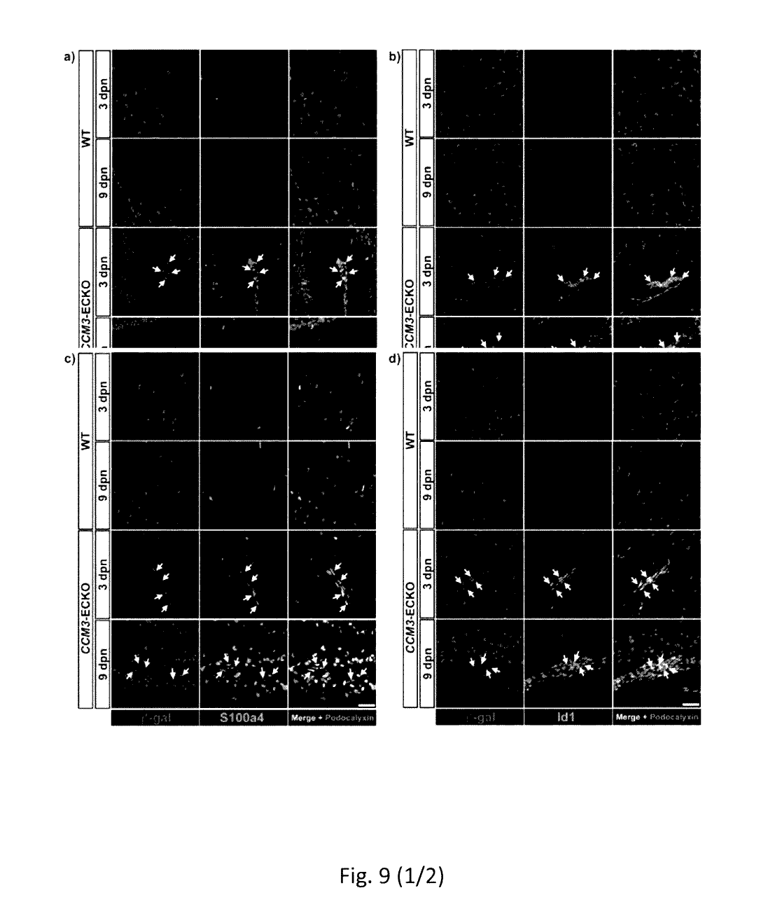

FIG. 8. Brain endothelial cells in CCM3-ECKO mice show enhanced β-catenin-mediated transcription in lesions of any size as well as in pseudo-normal vessels while TGF-β/BMP signaling is detectable only in larger lesions. a. Representative immunostaining for β-gal (red, upper panel) and p-Smad1 (phosphoSer463/465) (red, lower panel) in endothelial cells (Podocalyxin-positive, green) in brain sections from wild-type (WT) and CCM3-ECKO mice at 9dpn. Nuclei were stained with DAPI (blue). Pseudo-normal vessels as well as vascular lesions of increasing size are shown in CCM3-ECKO. Arrows, p-Smad1- or β-gal-positive nuclei; empty arrows, p-Smad1-negative nuclei. Scale bar, 50 μm. Similar results were obtained using p-Smad3 antibody (unpublished results) b. and c. Percentage of β-gal-positive or p-Smad1-positive endothelial nuclei on the total number of at least 250 endothelial nuclei counted for each condition. β-gal- and p-Smad1-positive and endothelial cell nuclei were counted in twenty random fields at 63× magnification in brain sections from matched littermate CCM3-ECKO (five) and WT (five) mice. *, p<0.05, t-test.

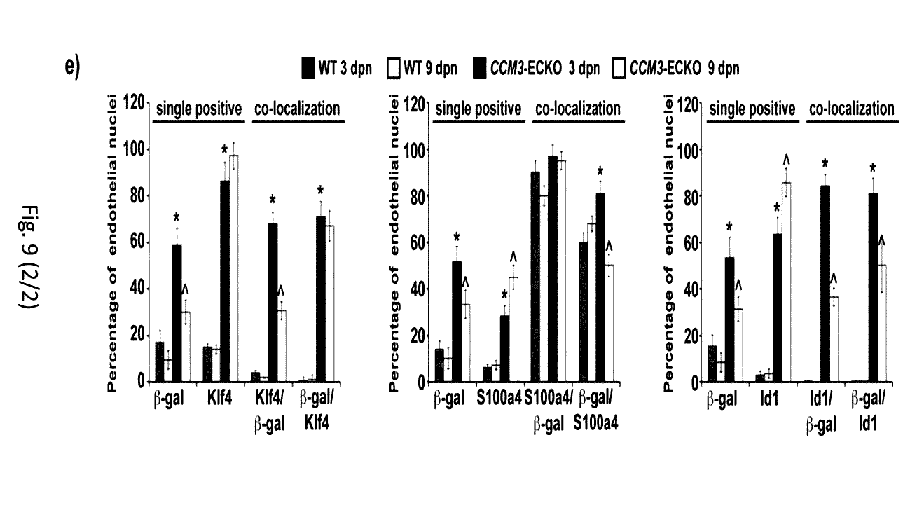

FIG. 9. Brain endothelial cells in CCM3-ECKO mice express stem-cell/EndMT markers in association with enhanced β-catenin transcription activity, a, b, c, d. Representative immunostaining of brain sections from wild-type (WT) and CCM3-ECKO mice, for β-gal (red) in combination with Podocalyxin (blue, to identify endothelial cells) and different stem-cell/EndMT markers (Klf4, Ly6a, S100a4, Id1, all green), at 3dpn (day post-natal) and 9dpn after CCM3 recombination at 1dpn. Arrows point to endothelial nuclei (Podocalyxin positive cells) expressing both β-gal and stem-cell/EndMT markers (see Merge, right column in each panel, yellow). Scale bar, 40 μm. e. Quantification of endothelial nuclei positive β-gal, Klf4, S100a4 and Id1 (single positive), and of their co-localization in brain sections of WT and CCM3-ECKO pups at 3 and 9dpn. For co-localization of each stem-cell/EndMT marker with β-gal the inventors distinguished two populations of endothelial cells: the β-gal positive one within which the inventors counted the number of EndMT positive nuclei and the EndMT positive one within which the inventors counted the number of β-gal positive nuclei. This analysis shows that Klf4, S100a4 and Id1 expression is highly linked to β-catenin transcription activity in 3dpn pups, while it becomes partially uncoupled in 9dpn pups.

A total number of at least 600 nuclei was counted in fifty random fields at 63× magnification for each condition in samples from matched littermate pups in three independent experiments. *, p<0.05 versus respective WT values (t-test); ̂, p<0.05 versus value in 3dpn CCM3-ECKO pups.

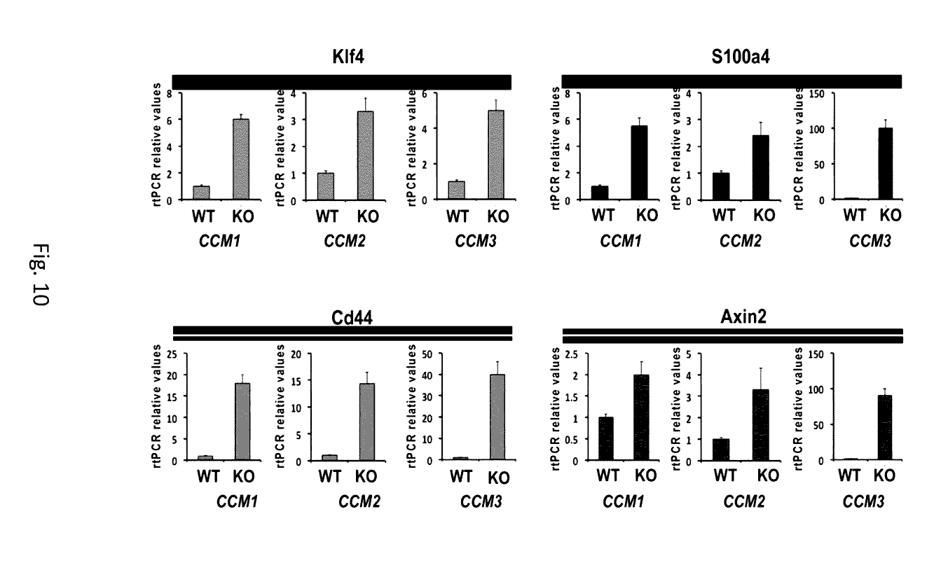

FIG. 10. Induction of End-MT markers and β-catenin target genes in CCM deficient endothelial cells in culture. Deletion of either CCM1 or CCM2 or CCM3 in cultured endothelial induces activation of b-catenin-driven transcription, as indicated by enhanced transcription of axin2 in comparison to respective WT. EndMt markers (Klf4, Cd44 and S100a4) are also upregulated Figures reports rtPCR

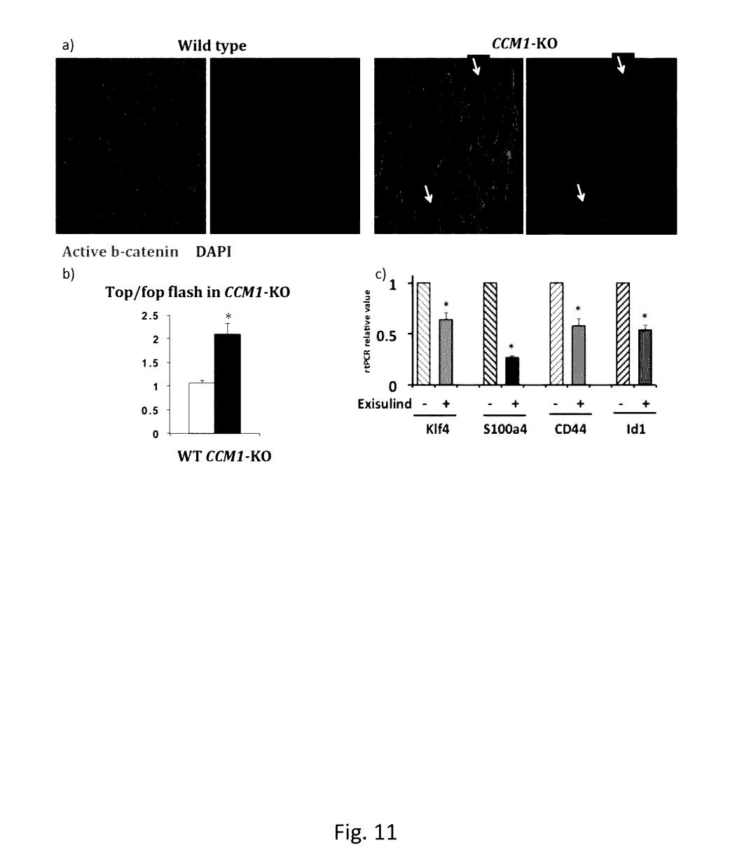

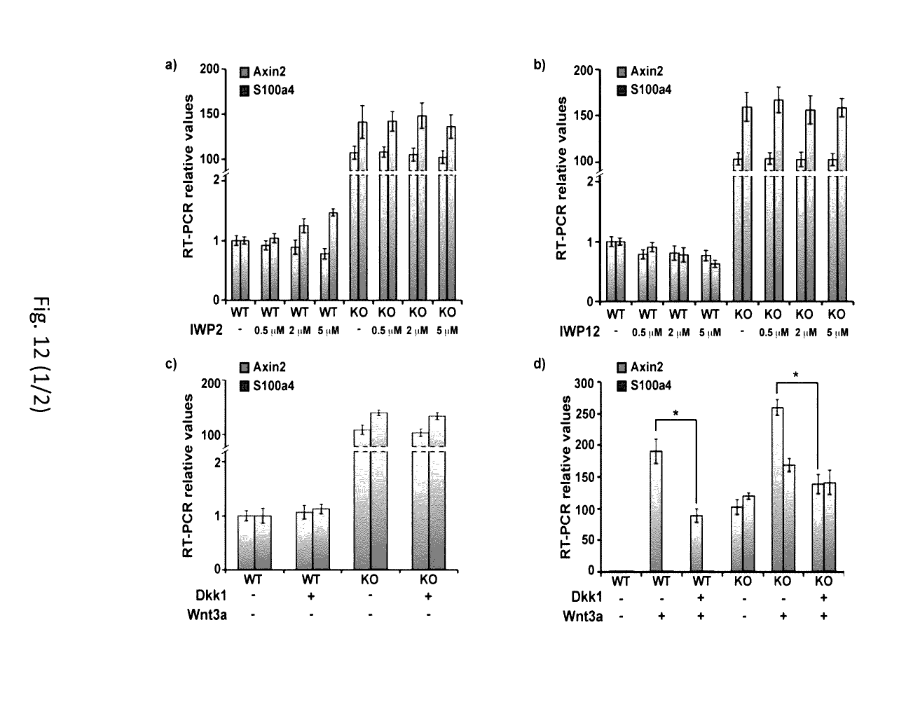

FIG. 11. Nuclear β-catenin is transcriptionally active in CCM1-KO endothelial cells and activates the transcription of EndMT markers. a. Active β-catenin (dephosphorylated on residues 37/41) concentrates in the nuclei of CCM1-KO endothelial cells in culture (arrows), while it is absent from the nuclei and localizes to cell-to-cell contacts in wild-type endothelial cells. b. Nuclear β-catenin is transcriptionally effective in CCM1-KO endothelial cells as indicated by enhanced transcriptional response versus WT in the Top-fop flash assay. c. β-catenin contributes to the expression of EndMT markers in CCM1-KO endothelial cells as indicated by exisulind. inhibition *, p<0.05 versus respective solvent-treated value (t-test).

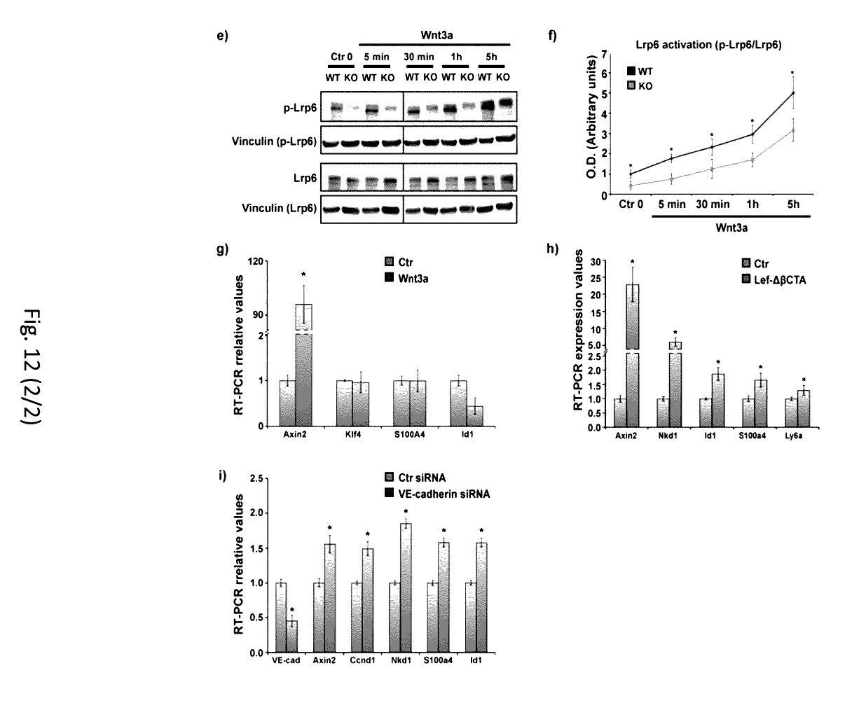

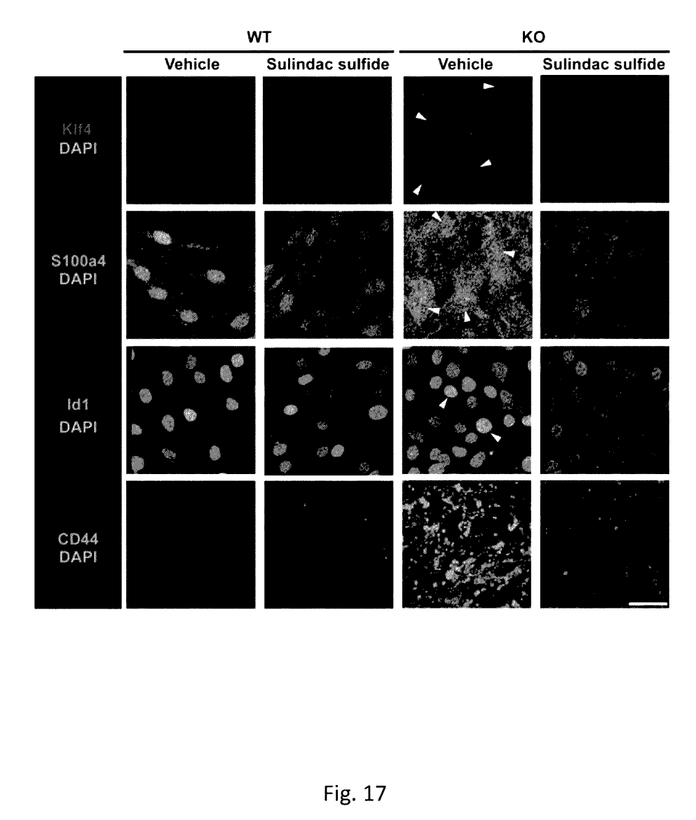

FIG. 12. CCM3-knockout endothelial cells show cell-autonomous, Wnt-receptor independent activation of β-catenin signaling, a, b, c, d. β-catenin-driven activation of both Axin2 and S100a4 transcription in un-stimulated CCM3-knockout (KO) endothelial cells was not inhibited by either the porcupine inhibitors IWP2 or IWP12 (a, b) nor by the Lrp competitor Dkk1 (0.5 uM) (c) that effectively inhibited Wnt3a-induced stimulation of Axin2 both in wild-type and CCM3-knockout endothelial cell lines (d). Transcription of S100a4 was neither induced by Wnt3a in wild-type cells nor inhibited by Dkk1 in CCM3-knockout cells. e, f. The Wnt co-receptor Lrp6 is less activated (phosphorylated) both in basal condition and after Wnt3a stimulation in CCM3-knockout in comparison to wild-type endothelial cells. Western blot representative of three independent experiments, quantified in (f), is shown. *, p<0.05 versus WT (t-test). g, h. Acute stimulation (48 h) with Wnt3a cannot induce expression of stem-cell/EndMT markers in wild-type endothelial cells, while sustained stimulation (7 days), by-passing Wnt receptor, with constitutively active form of beta-catenin Lef-Δ8CTA (Vleminckx et al, 1999) does. *, p<0.05 versus WT (t-test). i. Activation of typical β-catenin target genes (Axin2, Ccnd1, Nkd1) and stem-cell/EndMT markers (S100a4, Id1) is an early response to VE-cadherin silencing by siRNA (48 h) in wild-type endothelial cells. For analogous acute response to knockdown of CCM3 by siRNA see FIG. 17. *, p<0.05 versus negative control siRNA-treated cells (t-test).

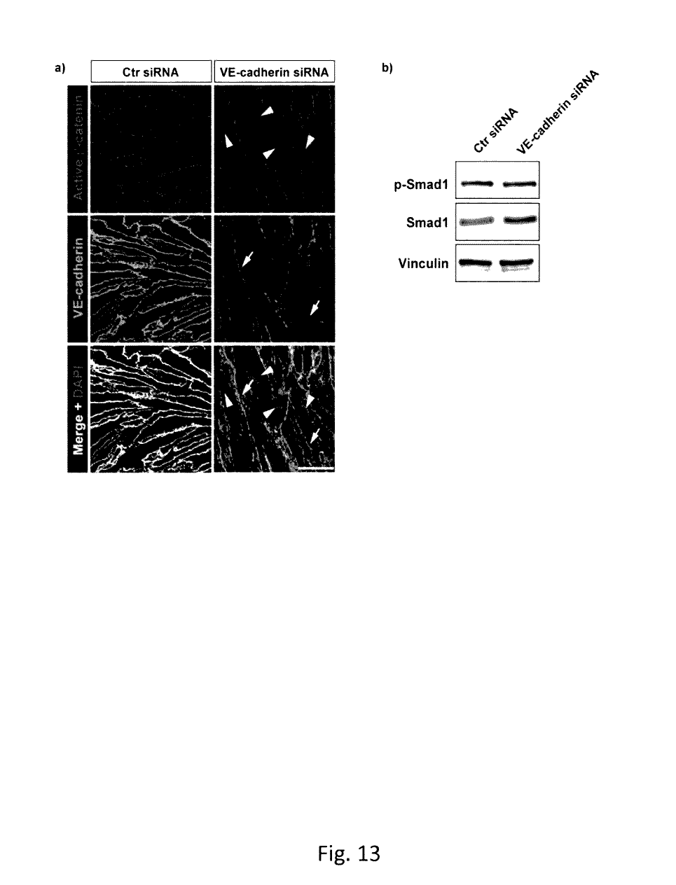

FIG. 13. Junction dismantling following VE-cadherin silencing induces nuclear accumulation of active β-catenin in endothelial cells. However, Smad1 phosphorylation is not enhanced. a. Representative immunostaining of active-β-catenin (red) and VE-cadherin (green) after VE-cadherin acute down regulation (48 h) through siRNA in wild-type endothelial cell line. Junction dismantling and VE-cadherin down regulation (arrows) is accompanied by nuclear accumulation of active-β-catenin (arrowheads. DAPI indicating nuclei is in blue). Controls (Ctr siRNA) were treated with negative (not-targeting) siRNA. Scale bar, 30 μm. b. In the same cells VE-cadherin knockdown did not stimulate phosphorylation of Smad1 measured in Western blot.

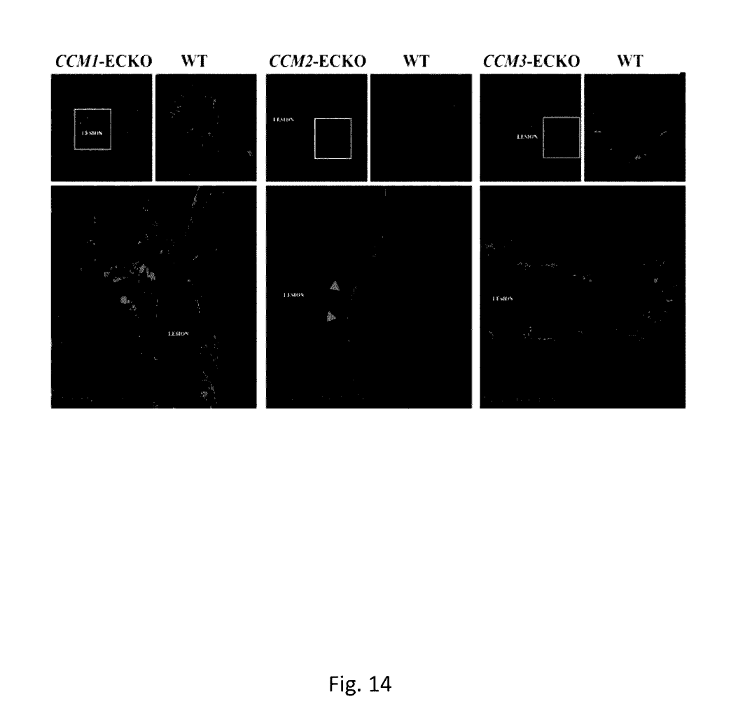

FIG. 14. A common feature of CCM1, 2, and 3 deletion is junction dismantling. Endothelial cells lining vascular cavernomas (LESION) show disorganized adherens junctions (VE-cadherin staining, red). Brain sections of CCM1-ECKO, CCM2-ECKO and CCM3-ECKO pups (7dpn and with CCM gene ablation at 1dpn) are shown. In comparison, VE-cadherin is regularly distributed to cell-to-cell contacts in brain endothelial cells of wild type (WT) littermates. Nuclei are blue by DAPI. Yellow boxed areas in sections from CCM-ECKO brains are magnified in the bottom panels.

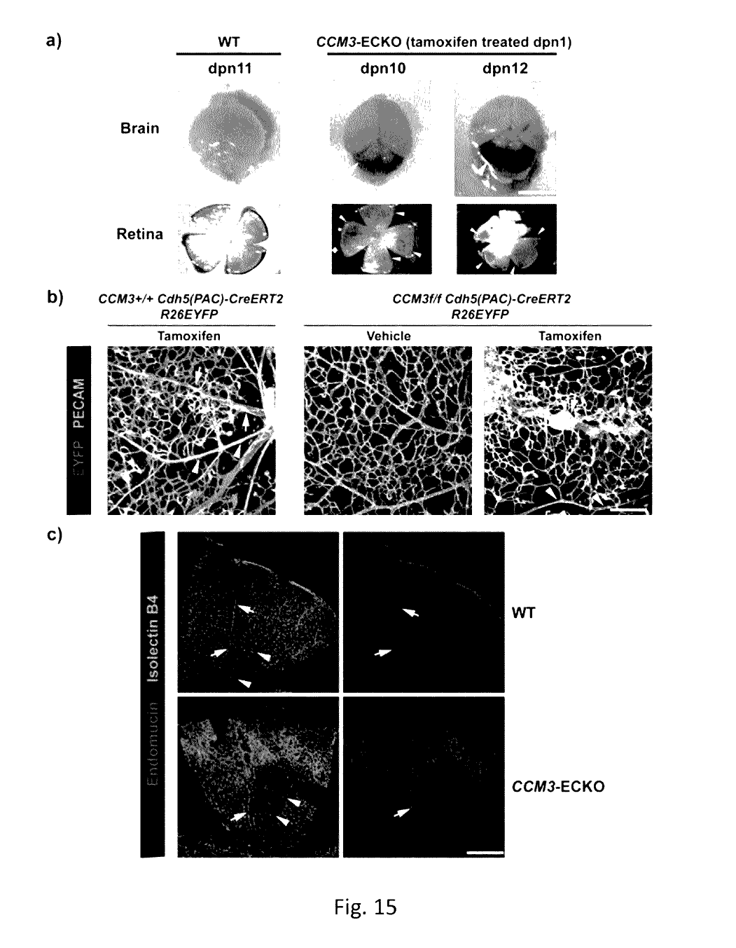

FIG. 15. After endothelial-selective expression of Cre-recombinase in CCM3-flox/flox-Cdh5(PAC)-CreERT2 mice (for tamoxifen-inducible endothelial-cell-specific expression of Cre-recombinase and CCM3 gene recombination), the brain and retina of this mouse model show the formation of vascular lesions. These malformations develop from the venous vessels, even though Cre recombinase is also active in the arteries. a. The CCM3-flox/flox-Cdh5(PAC)-CreERT2 mice were treated with tamoxifen (10 mg/kg body weight, as described in Methods) at dpn1 to induce endothelial-cell-selective expression of Cre recombinase and recombination of the floxed/floxed CCM3 gene (CCM3-ECKO mice). The macroscopic appearance following dissection showed evident lesions in the cerebellum and retina (arrowheads). In the brain, some superficial vascular malformations can also be observed (small arrowheads), but most lesions can only be detected after sectioning and immunostaining, as shown in the main text. These lesions began to appear three to four days after treatment with tamoxifen, and they progressively increased in size. From 10 days after tamoxifen treatment, these CCM3-ECKO mice started to die, with evident hemorrhagic cerebellum. Tamoxifen did not induce any phenotype both in CCM3-floxed/floxed-Cdh5(PAC) mice negative for CreERT2, that did not express Cre recombinase, and in the heterozygous CCM3-floxed-Cdh5(PAC)-CreERT2 mice. The wild-type (WT) mice were CCM3⁺/⁺-Cdh5(PAC)-CreERT2 mice treated with tamoxifen. CCM3-floxed/floxed-Cdh5(PAC)-CreERT2 mice treated with the vehicle used to dissolve tamoxifen also showed a WT phenotype. Scale bar, 1 cm. b. CCM3-flox/flox-Cdh5(PAC)-CreERT2 mice were bred with Rosa 26-Enhanced Green Fluorescent Protein (EYFP) mice (Srinivas et al, 2001) to monitor the expression of Ore-recombinase through the expression of EYFP. In vessels of the retina from CCM3^{+/+}-Cdh5(PAC)-CreERT2-R26-EYFP mice and CCM3-flox/flox-Cdh5(PAC)-CreERT2-R26-EYFP (CCM3-ECKO) mice, Ore-induced recombination (with tamoxifen treatment, as above) is indicated by the expression of the reporter gene EYFP. This was frequent in arteries (arrowheads), veins (arrows) and microvessels of both of these mouse models, and was seen as extensive co-localisation of EYFP and Pecam (marker of endothelial cells) labeling. The CCM3 transcript was reduced by more than 80%, as assessed by rtPCR in freshly isolated brain microvessels of CCM3-ECKO pups, in comparison to the vehicle-treated wild-type mice. Scale bar, 200 mm. c. In the retina of the CCM3-flox/flox-Cdh5(PAC)-CreERT2 (CCM3-ECKO) mice, malformations only developed on the venous side of the vascular network, which can be distinguished morphologically in the retina (as in b.) and by endomucin-positive staining (arrows). Arrowheads indicate arterial vessels, which are endomucin negative and isolectin B4 positive. The inventors have also observed a similar venous-specific defect after endothelial-specific ablation of both CCM1 (Maddaluno, et al, 2013) and CCM2 (Boulday et al, 2011). Scale bar, 700 mm. In b and c retinas from dpn9 littermate mouse pups are shown.

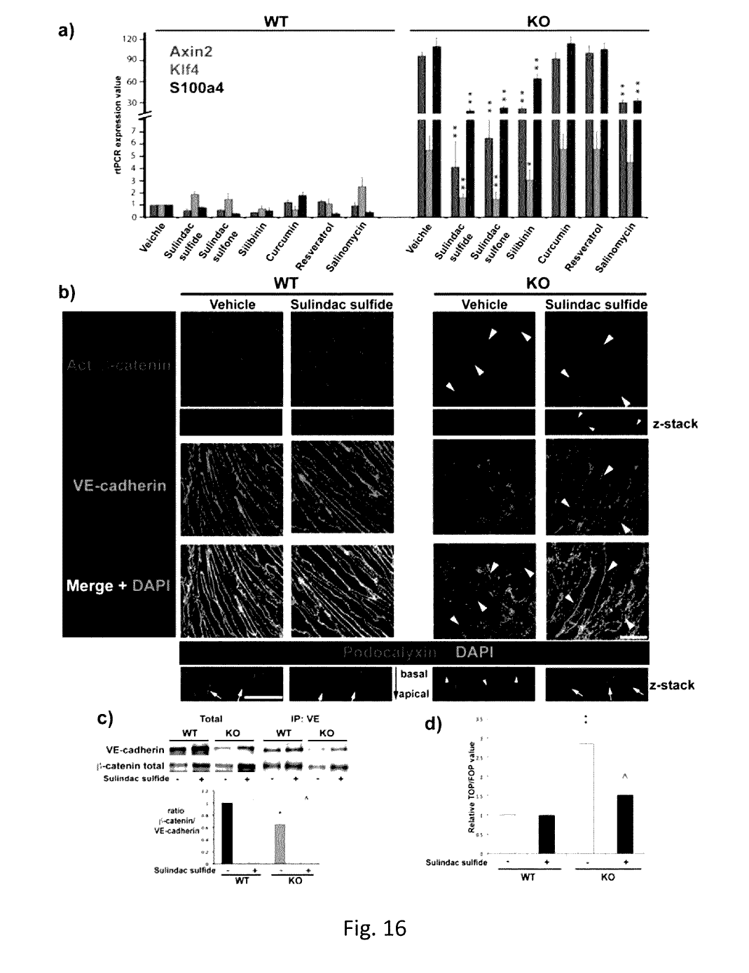

FIG. 16. CCM3-knockout endothelial cell line shows sulindac sulfide inhibition of transcription of b-catenin target genes and progenitor/EndMT markers (a), inhibition of localization of active b-catenin to the nucleus and induction of association to adherens junctions together with VE-cadherin (b), inhibition of the loss of the co-immunoprecipitation complex of b-catenin and VE-cadherin (c), and inhibition of b-catenin/Tcf4-dependent transcription of the luciferase reporter gene in the Top/Fop flash assay (d). a. Quantification of effects of sulindac sulfide, sulindac sulfone, and other drugs that have been reported to target different steps of the b-catenin signaling pathway, on the overexpression of a b-catenin target gene (Axin2) and endothelial progenitor/EndMT markers (Klf4, S100a4) in these wild-type (WT) and CCM3-knockout endothelial line. Sulindac sulfide and sulindac sulfone show the greatest inhibition of the strong induction of transcription of Axin2, Klf4 and S100a4 seen in these CCM3-knockout endothelial cells. Among the other drugs tested here, silibinin was effective against these three transcripts, while salinomycin significantly reduced only Axin2 and S100a4. Data are mean rtPCR values from at least three independent experiments, each carried out in triplicate. *, p<0.05, **, p<0.01 versus the respective transcripts in the vehicle-treated CCM3-knockout endothelial cells (t-test). See Methods for further details. Comparable results were obtained with cells from between five and 25 passages after knockout of CCM3. b. Representative immunostaining for effects of sulindac sulfide on re-localization of active b-catenin from the adherens junctions into the nucleus in these wild-type (WT) and CCM3-knockout endothelial cell line. Active b-catenin and VE-cadherin were lost from cell-cell contacts (adherens junctions) in these CCM3-knockout endothelial cells (top main panels, XY axis; small lower panels, Z projection along X axis). The active b-catenin (KO vehicle, arrowheads) is concentrated into the nucleus (see corresponding arrowheads in DAPI stainings) (right panels). Treatment with sulindac sulfide restored the distribution of active b-catenin to the adherens junctions (right panels, sulindac sulfide, arrowheads, and small lower right panels, sulindac sulfide, small arrowheads, for distribution along Z axis) and co-localization of active b-catenin and VE-cadherin is observed (arrowheads). Bottom panels of Z projection along X axis: a marker of apical polarity in endothelial cells, podocalyxin, shows loss of apical polarity in these CCM3-knockout endothelial cells. Podocalyxin is re-localized from the apical surface (left panels, WT, arrows) to be ectopically distributed on the basal side with CCM3 knockout (right panel, vehicle, arrowheads), as it has been reported for CCM1 knockout⁴⁶. Nuclei are outlined by white lines (DAPI). Sulindac sulfide re-establishes the correct apical distribution of podocalyxin (right panel, sulindac sulfide, arrows). Scale bar, 30 mm. c. Representative Western blotting (top) and quantification (bottom) of the effects of sulindac sulfide on the co-immunoprecipitation complex of b-catenin and VE-cadherin. Top: Western blotting with the wild-type (WT) and CCM3-knockout endothelial cells of total extracts and immunoprecipitates with VE-cadherin antibodies (IP: VE). With sulindac sulfide treatment, the reduction in the level of VE-cadherin in the CCM3 knockout was restored (compare in total sulindac sulfide-KO and vehicle-WT). Bottom: With the co-immunoprecipitation complex measured as the b-catenin/VE-cadherin ratio, with sulindac sulfide treatment, the significantly reduced association between b-catenin and VE-cadherin in the CCM3 knockout (35%±0.32 SD, *, p<0.05, vehicle-KO versus vehicle-WT) was restored to the level observed in the wild-type cells (WT) (̂, p<0.05, sulindac sulfide-KO versus vehicle-KO, t-test). The quantification of the bands from the Western blotting was assessed as the means of three independent experiments, using ImageJ. d. Sulindac sulfide inhibited (̂, p<0.05, sulindac sulfide-KO versus vehicle-KO, t-test) the significant increase (**, p<0.01, vehicle-KO versus vehicle-WT, t-test) of b-catenin/Tcf4-dependent transcription of the luciferase reporter gene in the Top/Fop Flash assay (see Methods for details). The ratio between Top-flash and Fop-flash values normalized over transfection efficiency (b-galactosidase activity) is shown as fold change in comparison to the ratio in vehicle-WT (relative Top/Fop-flash value).

FIG. 17. CCM3-knockout endothelial cell line shows sulindac sulfide inhibition of overexpression of endothelial progenitor/EndMT²⁰ markers. Representative immunostaining showing that compared to the wild-type (WT), there was increased expression of Klf4, S100a4, Id1 and CD44 in these CCM3-knockout (KO) endothelial cells. Nuclei were stained with DAPI (outlined by white lines). The overexpression of Klf4 (top row) and Id1 (third row) in CCM3 knockout was confined to the nucleus (arrowheads to white nuclei in KO, vehicle). Similarly, S100a4 (second row) was both nuclear (KO, vehicle, white nuclei, arrowheads) and cytoplasmic (KO, vehicle), while CD44 (bottom row) was mostly cytoplasmic (KO, vehicle). Treatment with sulindac sulfide (right-hand panels) strongly reduced the overexpression of these proteins in these CCM3-knockout (KO) endothelial cells. Comparable results were obtained in primary cultures of CCM3-knockout brain endothelial cells, as shown in FIG. 3. Scale bar, 30 mm.

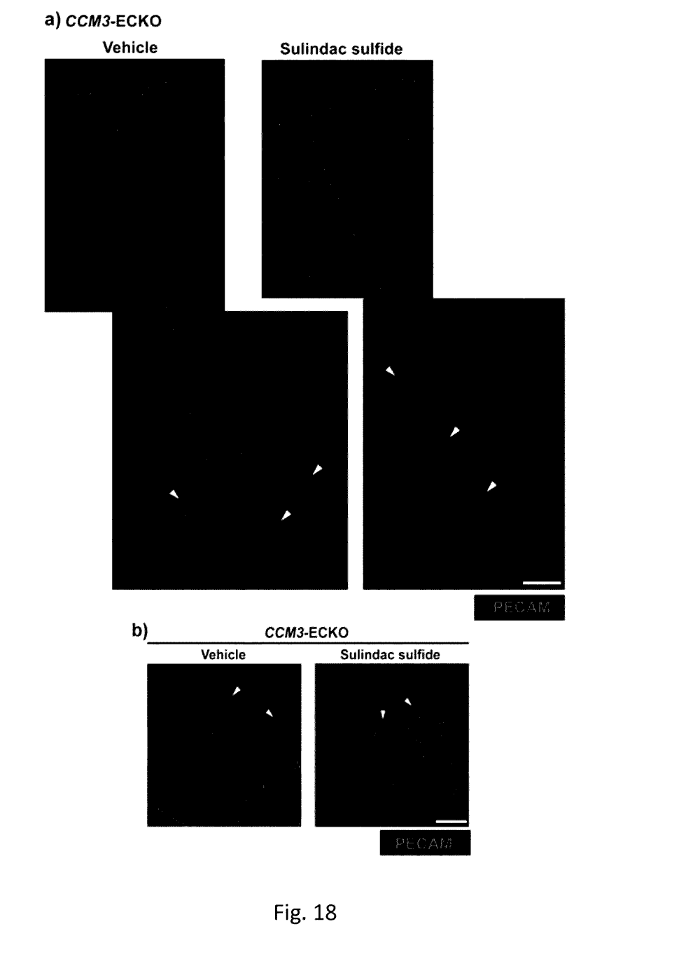

FIG. 18. CCM3-ECKO pups (at dpn9) treated (from dpn2) with vehicle or sulindac sulfide, as described in the Methods, show sulindac sulfide reduction of the malformations in cerebral vessels. Representative immunostaining of brain sections with Pecam (endothelial cells). a. Vessels of the superior sagittal sinus that enters the brain from the dorsal surface. With the CCM3 knockout (Vehicle), the straight vessels with large diameters are seen to terminate in budding branches that form cavernae. In a comparable vessel, sulindac sulfide treatment in this CCM3 knockout greatly reduces the diameters of these vessels and promotes apparently normal terminal branching. The panels show maximal projections of confocal optical sections of samples acquired at 20× magnification. Scale bar, 100 mm. b. Lesions in internal sagittal sections of the CCM3-ECKO pups treated as in a. With the CCM3 knockout (vehicle), the terminal regions of the Great Cerebral vein of Galean show mulberry lesions that appear to form at the terminus of branching vessels (arrowheads). Sulindac sulfide treatment in this CCM3 knockout greatly reduces the mulberry budding ends. Scale bar, 100 mm.

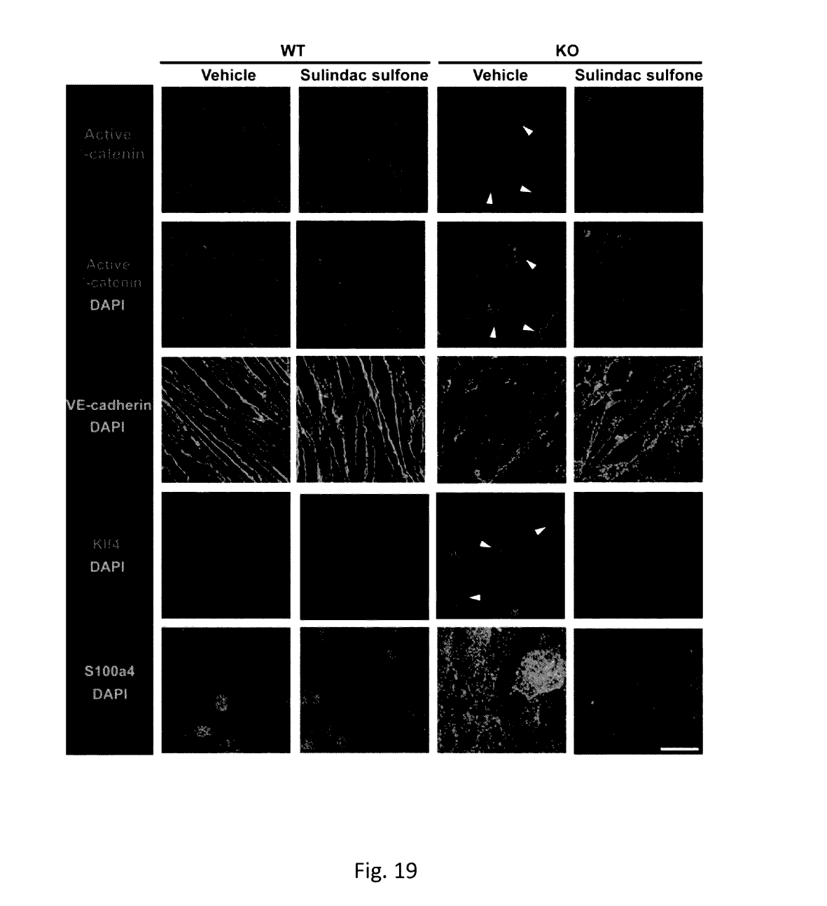

FIG. 19. Similar to the effects of sulindac sulfide, CCM3-knockout endothelial cell line show sulindac sulfone inhibition of loss of active b-catenin and VE-cadherin from cell-cell contacts (adherens junctions), of accumulation of active b-catenin in the nucleus, and of overexpression of progenitor/EndMT markers. Representative immunostaining showing that the loss of active b-catenin from cell-cell contacts (top row) and its relocalization to the nucleus (second row) in these CCM3-knockout endothelial cells (KO, Vehicle, arrowheads) was inhibited by sulindac sulfone treatment. Nuclei were stained with DAPI. Similarly, VE-cadherin loss from cell-cell contacts was inhibited by sulindac sulfone treatment (third row). Overexpression of Klf4 (nuclear, Vehicle, arrowheads) and S100a4 (cytoplasmic and nuclear) in these CCM3-knockout (KO) endothelial cells was also strongly reduced by sulindac sulfone treatment (bottom rows). Nuclei (DAPI) are outlined by white line in VE-cadherin, Klf4 and S100a4 stainings. Scale bar, 30 mm.

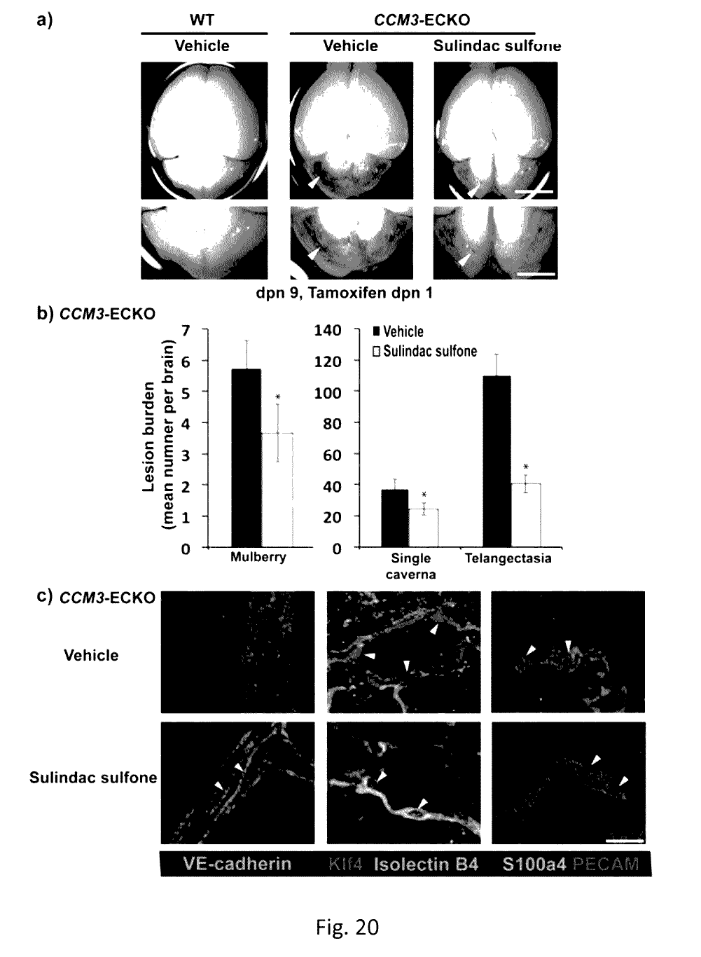

FIG. 20. Similar to the effects of sulindac sulfide CCM3-flox/flox-Cdh5(PAC)-CreERT2-BAT-gal (CCM3-ECKO) mice show sulindac sulfone reduction of the malformations in cerebral vessels. These mice were treated with tamoxifen (10 mg/kg body weight, as described in Methods) at dpn1 to induce endothelial-cell-selective expression of Ore recombinase and recombination of the floxed/floxed CCM3 gene (CCM3-ECKO mice). They were also treated with vehicle or with sulindac sulfone (30 mg/kg) daily, starting from dpn2. a. The macroscopic appearance of the dpn9 mouse pup brains following dissection showed evident lesions in the cerebellum of the CCM3-ECKO mice (arrowheads). Scale bar, 0.65 cm. Lower panels: Further magnification of the cerebellum. Scale bar, 0.3 cm. b. Quantification of mean brain lesions as the mulberry (multiple lumens), single caverna, or telangiectases lesions in the entire brains (as described in⁶², see Methods) from three vehicle-treated and three sulindac-sulfone-treated CCM3-knockout mice. The brains were sectioned along the sagittal axis (150 mm sections, vibratome), immunostained for Pecam (endothelial cells) and examined by wide-field fluorescence microscopy (10× and 20× magnification). *, p=0.0053, 0.006, 0.004 for mulberry, single caverna and telangiectase lesions, respectively (Wilcoxon test). c. Representative immunostaining of localization of VE-cadherin, Klf4 and S100a4. Left: From the diffuse state of VE-cadherin in vehicle-treated CCM3-ECKO mice, sulindac sulfone restored its localization to cell-cell junctions (arrowheads). Middle, right: From the nuclear staining for the expression of Klf4 (middle) and S100a4 (right) in vehicle-treated CCM3-ECKO mice, sulindac sulfone reduced this nuclear reactivity for both Klf4 and S100a4. Endothelial cells are stained by either Isolectin B4 or PECAM. Scale bar, 15 mm.

### DETAILED DESCRIPTION OF THE INVENTION

### Methods

### Endothelial-Cell-Specific Recombination in CCM3-Flox/Flox Mice to Generate CCM3-ECKO Mice

The CCM3-flox/flox mice were generated at TaconicArtemis (Koeln, Germany). Two P-lox sequences were inserted that flank exons 4 and 5 of the murine CCM3 gene, to produce a loss-of-function mutation after excision by Ore recombinase. These CCM3-flox/flox mice were bred with Cdh5(PAC)-CreERT2 mice (Wang et al, 2010) for tamoxifen-inducible endothelial-cell-specific expression of Ore-recombinase and CCM3 gene recombination. The CCM3-flox/flox-Cdh5(PAC)-CreERT2 mice were further bred with BAT-gal mice (Maretto et al, 2003) to monitor the activation of b-catenin transcription signaling, and with Rosa 26-Enhanced Green Fluorescent Protein (EYFP) (Rosa26EYFP) mice (Srinivas et al, 2001) to monitor the expression of Ore-recombinase through the expression of EYFP. Tamoxifen (Sigma) was dissolved in corn oil and 10% ethanol (at 10 mg/ml), and then diluted 1:5 in corn oil before single intragastric administration to dpn 1-2 pups (35 mg/kg body weight), as described in¹⁴. The control (wild-type) mice included CCM3-flox/flox-Cdh5(PAC)-CreERT2-BAT-gal mice treated with the vehicle used to dissolve the tamoxifen (corn oil plus 2% ethanol), and CCM3^{+/+}-Cdh5(PAC)-CreERT2-BAT-gal mice treated with tamoxifen.

CCM1-ECKO and CCM2-ECKO have been obtained from CCM1-flox/flox and CCM2-flox/flox mice as described in details above for CCM3-ECKO mice, see also Maddaluno et al, 2013 and Boulday et al, 2011.

### Mouse Genotyping

The following probes were used for the mouse genotyping: wild-type CCM3 allele: 5′ GAT AGG AAT TAT TAO TGC CCT TCC 3′ (SEQ ID No. 1), 5′ GAO AAG AAA GCA CTG TTG ACC 3′ (SEQ ID No. 2); deleted CCM3 gene after recombination induced by Cre recombinase: 5′ GAT AGG AAT TAT TAO TGC CCT TCC 3′ (SEQ ID No. 3), 5′ GCT ACC AAT CAG CTT CTT AGC CC 3′ (SEQ ID No. 4), Cdh5(PAC)-CreERT2 gene: 5′ CCA AAA TTT GCC TGC ATT ACC GGT CGA TGC 3′ (SEQ ID No. 5), 5′ ATC CAG GTT ACG GAT ATA GT 3′ (SEQ ID No. 6); BAT-gal gene: 5′ CGG TGA TGG TGC TGC GTT GGA 3′ (SEQ ID No. 7), 5′ ACC ACC GCA CGA TAG AGA TTC 3′ (SEQ ID No. 8); Rosa 26 EYFP gene: 5′ GCG AAG AGT TTG TCC TCA ACC 3′ (SEQ ID No. 9), 5′ GGA GCG GGA GAA ATG GAT ATG 3′ (SEQ ID No. 10), 5′ AAA GTC GCT CTG AGT TGT TAT 3′ (SEQ ID No. 11).

Treatment with Sulindac Sulfide and Sulindac Sulfone

Sulindac sulfide (Sigma) and sulindac sulfone (Sigma) were both dissolved in DMSO and further diluted 1:50 in corn oil. They were administered intragastrically, daily (30 mg/kg body weight), starting one day after the induction of recombination. The control mice were treated in parallel with vehicle only (corn oil plus 2% DMSO). The inventors did not observe either increased bleeding from vascular lesions or mortality in drug-treated CCM3-ECKO mice in comparison to vehicle-treated ones.

Treatment with IWP2, IWP12, DKK1 and Wnt3a

Drugs were added to confluent cells for 48 h before the indicated assays. Final concentrations for IWP2 and IWP12, both from Sigma-Aldrich, were: 0.5, 2, 5 DM. Drugs were dissolved in DMSO, control treatment (Vehicle) was 0.1% DMSO final concentration, as for drug treatment. Murine recombinant Dkk1 (R&D) was 0.5 DM on cells. Murine recombinant Wnt3a (R&D) was 5 ng/ml for the time indicated in the figure legends.

In Vitro Isolation, Culture and Recombination of Endothelial Cells from the CCM3-Flox/Flox Mice

Endothelial cells from the CCM3-flox/flox mice (8-10 weeks old) were isolated from the brain as previously described¹³. Recombination of the floxed CCM3 gene was induced by treating the cells at culture day 1 with the AdenoCre viral vector, as previously described⁵⁹. The control endothelial cells were an aliquot of the same endothelial cell preparation treated with AdenoGFP, instead of AdenoCre. The cells were then maintained in culture for up to a further 7 days before analysis, as described in the main text. Drug treatments were for 48 h before the processing of the cells.

In some experiments, endothelial cell lines from the lungs of CCM3-flox/flox mice (8-10 weeks old) were immortalized in culture through retroviral expression of polyoma middle T gene⁶⁰. Ablation of the CCM3 gene was achieved with the AdenoCre viral vector (with AdenoGFP in the control cells). These cells were then maintained in culture for up to 25 passages without detectable changes in the effects of this CCM3 ablation. These endothelial cell lines responded to the absence of CCM3 in a comparable way to both primary cultures of brain endothelial cells in vitro and brain endothelial cells in vivo.

### Drug Treatment of Cells in Culture

Drugs were added to confluent cells for 48 h before the indicated assays. The final concentrations used were: 135 mM sulindac sulfide, 125 mM sulindac sulfone, 200 mM silibinin, 40 mM curcumin, 40 mM resveratrol, and 250 mM salinomycin (all from Sigma-Aldrich). As all of these were dissolved in DMSO, control treatment (vehicle) was 0.1% DMSO final concentration, as for drug treatment.

### Immuno-Staining for Fluorescence Microscopy of Brain Sections, Retinas and Cells in Culture

Brains and eyes from mice pups were fixed in 3% paraformaldehyde immediately after dissection, and this fixing was continued overnight at 4° C. The retinas were dissected from the eyes just before staining as the whole mount. Fixed brains were embedded in 4% low-melting-point agarose and sectioned along the sagittal axis (150 mm) using a vibratome (1000 Plus, The Vibratome Company, St. Louis, Mo., US).

Brain sections and retinas were stained as floating samples in 12-well and 96-well plates, respectively. They were blocked overnight at 4° C. in 1% fish-skin gelatin with 0.5% Triton X100 and 5% donkey serum in phosphate-buffered saline (PBS) containing 0.01% thimerosal. The samples were incubated overnight at 4° C. with the primary antibodies diluted in 1% fish-skin gelatin with 0.25% Triton X100 in PBS containing 0.01% thimerosal. Following washing with 0.1% Triton X100 in PBS, the secondary antibodies were added for 4 h at room temperature in 1% fish-skin gelatin with 0.25% Triton X100 in PBS containing 0.01% thimerosal. The incubation with DAPI was in PBS for 4 h, which was followed by several washes in PBS, post-fixating with 3% paraformaldehyde for 5 min at room temperature, and further washes in PBS. The brain sections were mounted in Vectashield with DAPI, and the coverslips fixed with nail varnish; the retinas were mounted in Prolong gold with DAPI.

Cells cultured in vitro were fixed and stained as described previously⁴⁶.

### Antibodies

The following antibodies were used: anti-Pecam (hamster; MAB1398Z, Millipore); anti-b-galactosidase (chicken; ab9361, Abcam); anti-VE-cadherin (rat monoclonal; 550548, BD Biosciences); anti-VE-cadherin (goat; sc-6458, Santa Cruz); anti-active-b-catenin (mouse monoclonal clone 8E7) dephosphorylated on Ser37 and Thr41, Millipore); anti-total-b-catenin (mouse monoclonal; Cell Signaling); anti-S100a4 (rabbit; 07-2274, Millipore); anti-Klf4 (goat; AF3158, R&D); anti-CD44 (rat; 553131, BD Biosciences); anti-ID1 (rabbit; sc-488, Santa Cruz); anti-aSMA (mouse monoclonal; F3777, Sigma); anti-GFP (rabbit; A-6455, Invitrogen); anti-podocalyxin (goat; AF1556, R&D); anti-phospho-histone H3 (rabbit; ab51776, Millipore); anti-CCM3 (rabbit; Eurogentec); anti-p-Smad1 (rabbit; 9516, Cell Signaling); anti-endomucin (rabbit; sc-65495, Santa Cruz); anti-α-tubulin (mouse monoclonal; T9026, Sigma). Biotin-conjugated isolectin B4 (Vector Lab), revealed with Alexa555-conjugated streptavidin (Molecular Probes), was also used to identify endothelial cells in retina and brain sections.

The secondary antibodies for immunofluorescence were anti-Alexa448 and anti-Alexa555, and Cy3-conjugated antibodies raised in the donkey against immunoglobulin of the appropriate animal species (Molecular Probes or Jackson Laboratories).

The secondary antibodies for Western blotting were HRP-linked anti-mouse, anti-rat and anti-rabbit antibodies (Cell Signaling), and HRP-linked anti-goat antibodies (Promega)

rtPCR

RNA extraction was performed with RNeasy kits (74106; Promega). The RNA (1 μg) was reverse transcribed with random hexamers (High Capacity cDNA Archive kits; Applied Biosystems). The cDNA was amplified with TaqMan gene expression assays (Applied Biosystems) using a 7900 HT thermocycler (ABI/Prism). For each sample, the expression levels were determined with the comparative threshold cycle (Ct) method, and normalized to the housekeeping genes encoding 18S and glyceraldehyde-3-phosphate dehydrogenase (GAPDH). For amplification, the following probes (Applied Biosystems) that have been validated to recognize the mouse transcripts in rtPCR were used: Axin2, Nkd1, Lef1, ccnd1, cMyc-Klf4, Ly6a, S100a4, Id1, Cdh2, Acta2, CD44. The probes to identify the CCM3 mRNA transcript were custom designed, as: forward, CGAGTCCCTCCTTCGTATGG (SEQ ID No. 12); reverse, GCTCTGGCCGCTCAATCA (SEQ ID No. 13); reporter sequence, CTGATGACGTAGAAGAGTACA (SEQ ID No. 14).

### Top/Fop-Flash Assay

For the detection of b-catenin-dependent transcription of a reporter target, the Top-Flash plasmid was used (0.3 mg/cm² cell culture area), which contains seven Tcf/Lef binding sites that control the transcription of firefly luciferase (Lluis et al, 2008). This was transfected into the endothelial cells from lung using Lipofectamine 2000, according to the manufacturer instructions (Invitrogen). The pCMV plasmid for constitutive expression of b-gal was co-transfected (0.1 mg/cm²), for normalization of luciferase expression over transfection efficiency. As the negative control, a Fop-Flash plasmid was used that contained six mutated (i.e., inactive) Tcf/Lef sites upstream of a minimal promoter and the firefly luciferase gene (0.3 mg/cm²). This was co-transfected with the b-gal plasmid, for normalization, as above. The Dual-Light Reporter Gene assay system (Applied Biosystems) for the combined detection of firefly luciferase and b-gal was used. The cell extraction and detection of chemiluminescence (Glomax 96 microplate luminometer; Promega) was carried out according to the manufacture instructions.

### Western Blotting and Immunoprecipitation

Standard procedures were used to extract and analyze the protein content by Western blotting and immunoprecipitation⁴⁶. Nuclear fractionation was as described previously⁶².

### Assessment of Lesion Burden

For the classification and counting of lesions, entire brains from dpn 9 littermate pups were sectioned and immunostained for Pecam as described above and in the Methods. Sections were then examined under wide-field fluorescence microscopy (10× and 20×). Lesions were classified as described in⁶³ as mulberry (multiple cavernae, group of more than two contiguous cavernae), single caverna (single dilated vessel with maximal diameter accommodating more than 25 red blood cells), or telangiectases, tortuous small vessels with abnormally dilated lumen). The total numbers of lesions were calculated by summing all of the types of lesions.

As the sections were 150-mm thick, a correction was applied to the number of mulberry lesions, which can span two sections. Therefore, the number of mulberry lesions was divided by 2.5. The lesions were counted and classified independently by two observers who were blinded to the treatments.

The maximal diameter of the mulberry lesions and single cavernae was used for statistical comparison.

siRNA Experiments

VE-cadherin expression was silenced using siRNA oligos to murine VE-cadherin (Smart pool, Thermo Scientific; target sequences: AGACAGACCCCAAACGUAA (SEQ ID No. 15), GAAAAUGGCUUGUCGAAUU (SEQ ID No. 16); AGGGAAACAUCUAUAACGA (SEQ ID No. 17); CCGCCAACAUCACGGUCAA (SEQ ID No. 18)), respectively, and Lipofectamine 2000 for transduction as described in Lampugnani et al, 2010.

### Statistical Analysis

Non-parametric Wilcoxon signed-rank tests were used to determine the statistical significance of the lesion burdens after the pharmacological treatments in vivo. Student's two-tailed non-paired t-tests were used to determine the statistical significance in the other in vitro and in-vivo analyses. The significance level was set at p<0.05.

### Results

Transcription Activity of b-Catenin is Enhanced In Vivo in Endothelial Cells of Endothelial-Cell-Specific CCM3-Knockout Mice

The in vivo mouse system presented here was initially generated through the cross of CCM3-floxed/floxed mice with Cdh5(PAC)-CreERT2 mice¹⁴, for tamoxifen-inducible endothelial-cell-specific expression of Cre-recombinase and CCM3 gene recombination. These mice were then further crossed with BAT-gal mice¹⁵, which show b-catenin-activated expression of the nuclear b-galactosidase (b-gal) reporter gene. As previously reported for CCM2⁶, these mice with endothelial-specific-inactivation of CCM3 gene induced postnatally (CCM3-ECKO) presented marked malformations and hemorrhages in the brain and retina vasculature comparable to CCM vascular lesions in patients. This was previously reported for CCM2 in an identical murine model⁶ and for CCM2 and CCM3 in a distinct murine system⁷. As CCM3 ECKO mice develop vascular malformations in the central nervous system this model provides a tool for testing pharmacological treatments as described below. For details of this experimental model, see Methods and FIG. 15.

Here, b-catenin-dependent transcription of the nuclear b-gal reporter gene is increased in vivo in endothelial cells of brain vessels of the newborn CCM3-ECKO, in comparison to matched control animals. As illustrated in the immunostaining in FIGS. 1a and 1b, the quantification by random-field counting using PECAM labeling of endothelial cells saw the b-gal-positive nuclei (0.86±0.15 positive nuclei per field; 7.2% positive nuclei of total 600 endothelial nuclei scored) of the control brain endothelial cells from the wild-type BAT-gal mice significantly increased by 4-fold in the CCM3-ECKO brain endothelial cells (5.0±1.7 positive nuclei per field; 36% positive nuclei of total 700 endothelial nuclei scored, p<0.05; t-test) (dpn 9 littermate pups). In these CCM3-ECKO brain endothelial cells, the b-gal-positive nuclei were distributed both in established caverns and in telangiectases (67% b-gal-positive endothelial nuclei in vascular lesions) and in pseudo-normal vessels (15% b-gal-positive endothelial nuclei in pseudo-normal vessels).

Expression of b-gal was also observed in endothelial cells of the retina of the CCM3-ECKO mice, in comparison with those from the control wild-type BAT-gal mice (FIG. 1c). Endothelial cells isolated from the brain of the CCM3-floxed/floxed mice (FIG. 2a, WT, primary culture) were compared with those after recombination of the CCM3 gene in vitro (CCM3-knockout brain endothelial cells; see Methods) (FIG. 2a, KO, primary culture), which showed active b-catenin in the nucleus (i.e., dephosphorylated on Ser37 and Thr41¹⁶). This effect paralleled strong alterations to the adherens junction organization that was seen as delocalization from the junctions of both active b-catenin (FIG. 2a) and VE-cadherin (FIG. 3c, vehicle). For the similar disorganization of VE-cadherin from endothelial junctions in vivo, see FIG. 4b, vehicle.

Due to limitations in both the supply of freshly isolated endothelial cells from the brain and their extremely limited mitotic index after the first in vitro passage detailed analyses of their b-catenin nuclear distribution and signaling were not possible. The inventors therefore established cultured endothelial cell lines where CCM3 was recombined in vitro as above¹⁷ (CCM3-knockout endothelial cell line) (see Methods). In these CCM3-knockout endothelial cell line, the inventors confirmed enhanced nuclear localization of b-catenin both by immunofluorescence (FIG. 2b) and cell fractionation (FIG. 2c).

In addition, beta-catenin and Tcf/Lef-dependent transcription of the exogenous luciferase gene was measured in Top/Fop Flash reporter assays. In these CCM3-knockout endothelial line, the transcription activity of b-catenin was significantly increased by 2.7-fold, compared to control wild-type cells (±0.2 SD; p<0.01) (FIG. 16d). Furthermore, the expression of some typical endogenous targets of b-catenin transcription activity was increased in these CCM3-knockout endothelial line (i.e., Axin2, Lef1, Ccnd1¹⁸) and inhibited by in vitro infection with the Adeno viral vector coding for a dominant-negative Tcf4¹⁷ (FIG. 2d).

The inventors also analyzed the expression of genes related both to acquisition/maintenance of endothelial progenitor phenotype and endothelial-to-mesenchymal transition, (EndMT)^{20, 21}, as b-catenin transcription signaling has been shown to regulate the processes of differentiation and epithelial-to-mesenchymal transition (EMT) in other cell types^{22,23,24.} In addition, EndMT was found to play a critical role in the development of the vascular lesions in a murine model of endothelial-specific CCM1 knockout (Maddaluno, L. et al., Nature 498 (7455):492, 2013). Transcription of Klf4^{25,26}, Ly6a^{27,28} and S100a4^{29, 20}, Id1^{30,31}, Cdh2²⁰, Acta2²⁰ were found to be significantly enhanced in CCM3-knockout endothelial cells as compared to control cells (FIG. 2e, endothelial line and FIG. 3b, primary culture). In addition, these increases were dependent on b-catenin transcriptional activity, as they were inhibited by dominant-negative Tcf4¹⁷, as above, (FIG. 2e). The enhanced transcription of these genes also corresponded to their increased protein expression (see vehicle-treated CCM3-knockout in FIGS. 17 and 19, endothelial line, and FIG. 3c, primary endothelial cells, and their up-regulation in vivo in vehicle-treated CCM3-ECKO brain endothelial cells in FIG. 5 and FIG. 20).

Thus, abrogation of CCM3 expression in endothelial cells leads to increase in b-catenin transcriptional activity and target gene expression both in vivo and in vitro. In the light of these data, the inventors investigated the effects of anti-b-catenin agents in vivo, on the brain vascular lesions of these CCM3-ECKO mice.

Sulindac Sulfide Reduces b-Catenin Transcription Activity and Expression of Endothelial Progenitor and EndMT Markers in Endothelial Cells from the CCM3-ECKO Mice

The inventors then initially tested a range of agents in their experimental model in vitro that have been described as affecting b-catenin signaling³² and, most importantly, are already in clinical use: sulindac sulfide, sulindac sulfone^{33,34}, silibinin³⁵, curcumin³⁶ and resveratrol^{37,38}. Salinomycin³⁹, a reported inhibitor of Wnt receptor signaling, was also included, although its use to date has been limited to experimental models.

Sulindac sulfide and sulindac sulfone were the most effective of these for inhibition of expression of endogenous targets of b-catenin transcription activity (see above) in the CCM3-knockout endothelial cell line (FIG. 16a). In addition, sulindac sulfide reduced b-catenin transcriptional activity as measured by the Top/Fop Flash reporter assays (FIG. 16d).

The inventors therefore further analyzed the effects of sulindac sulfide on the CCM3 null phenotype. In primary cultures of CCM3-knockout brain endothelial cells, the inventors found that sulindac sulfide effectively inhibited the expression of endogenous b-catenin target genes (FIG. 3a and see inhibition by dominant-negative Tcf4, as above, FIG. 3b). In parallel, sulindac sulfide strongly inhibited the nuclear localization of active b-catenin while increasing its concentration at cell-cell junctions (FIG. 3c). In parallel, VE-cadherin was also more localized to cell-cell contacts in these cells (FIG. 3c) and in CCM3-knockout endothelial cell line (FIG. 16b).

Consistently, by co-immunoprecipitation and Western blotting analysis sulindac sulfide restored the reduced association between b-catenin and VE-cadherin (minus 35%±0.32 SD, p<0.05) in CCM3-knockout endothelial line (FIG. 16c).

In parallel with inhibition of transcription of b-catenin target genes, sulindac sulfide also inhibited the overexpression of respective proteins in CCM3-knockout endothelial cells (FIG. 3c, primary culture, and FIG. 17, endothelial cell line).

Sulindac sulfide was then investigated in vivo in newborn mice after induction of CCM3-ECKO. The inventors found that treatment with sulindac sulfide inhibited the expression of nuclear reporter gene b-gal (FIG. 4a) and of b-catenin target genes (FIG. 5) in the endothelial cell of brain vasculature too. VE-cadherin also appeared to be better localized at endothelial cell-cell junctions in vivo in the brain vessels of newborn CCM3-ECKO mice treated with sulindac sulfide (FIG. 4b).

### Sulindac Sulfide Reduces Development of Vascular Lesions in the Brain and Retina of the CCM3-ECKO Mice

A crucial aspect of the present study is whether inhibition of b-catenin signaling by sulindac sulfide may also reduce the vascular lesions in CCM3-ECKO pups. The inventors found that, indeed, the mean number and dimension of vascular lesions were reduced by sulindac sulfide treatment. As illustrated in the immunostaining in FIG. 6a and quantified in FIG. 6b, the mean number (±SD) of vascular lesions per brain in the vehicle-treated CCM3-ECKO pups was 166.8±22, with 72.6±9 vascular lesions with sulindac sulfide treatment (p<0.005; non-parametric Wilcoxon signed-rank test) and the mean maximal diameter of mulberry lesions (±SD) in the vehicle-treated CCM3-ECKO pups was 386±56 mm and 244±38 mm with sulindac sulfide treatment (p<0.05, t-test). Sulindac sulfide treatment did not significantly reduce the maximal diameter of single cavernae.

Sulindac sulfide treatment also inhibited the vascular malformations in the retina of CCM3-ECKO mice. In these mice the retinas show multiple-lumen vascular lesions that are particularly concentrated at the periphery of the vascular network. Such lesions develop from veins, which are enlarged, although straight (compare vehicle for WT and CCM3-ECKO in FIGS. 6c and 6e and FIG. 15 for the venous marker endomucin). Sulindac sulfide partially normalized this aberrant vascular network in CCM3-ECKO mice (FIGS. 6c and 6d). In addition, the enlargement of the most internal tract of the veins that characterizes the retinas of these CCM3-ECKO mice was inhibited after sulindac sulfide (89.5±7.1 mm in vehicle-ECKO versus 35±7.8 mm in sulindac sulfide-ECKO, mean±SD of 30 measurements in 14 retinas each for WT and KO) (FIG. 6e and FIGS. 18a and 18b, for the effects of sulindac sulfide on vessel diameter and veins in the brain of CCM3-ECKO pups). At variance, arteries do not show this aberrant phenotype (FIGS. 6c and 6e).

The data reported above strongly suggest that sulindac sulfide may have a therapeutic activity in CCM patients. However, it has been reported that this drug inhibits cycloxygenease in platelets possibly increasing the risk of hemorrhage. The inventors therefore tested sulindac sulfone, which is devoid of anti-cyclooxygenase activity³³ and does not have an impact on coagulation response. As observed after sulindac sulfide treatment, sulindac sulfone also reduced the nuclear accumulation of active b-catenin and restored cell-cell junctions in cultured CCM3-knockout endothelial cell line (FIG. 19). In addition, sulindac sulfone inhibited the expression of b-catenin target genes, (see for instance Klf4 and S100a4 in FIG. 19), as did sulindac sulfide (see above). When tested in vivo, sulindac sulfone reduced the number of lesions in the brain of the CCM3-ECKO mice to a level comparable to sulindac sulfide (the mean number of lesions per mouse brain (±SD) in the untreated control was 153.5±28 and was reduced to 68.6±10 with sulindac sulfone treatment, p<0.01; non-parametric Wilcoxon signed-rank test; FIGS. 20a and 20b). In addition, in vivo, in the endothelium of brain vessels of the CCM3-ECKO mice, sulindac sulfone inhibited the expression of Klf4 and S100a4 (FIG. 20c). Similar results indicating that sulindac sulfone (exisulind) inhibits the formation of cavernoma lesions in the brain of CCM1-ECKO mice have also been obtained.

Kinetics of Activation of βeta-Catenin Signaling in Endothelial Cells in Brain Cavernomas of Ccm3-Ecko Mice

CCM3 ECKO murine model is first choice in proof of principle experiments, in particular testing the inhibitory activity of a drug as it develops the most serious phenotype as also observed in patients.

As reported in FIG. 7 βeta-catenin signaling is early enhanced in the lesion and such activation precedes the activation of TGF-β/BMP pathway that the inventors have reported to contribute to the maintenance of the CCM phenotype (Maddaluno et al, 2013).

The in-vivo mouse system used was generated through the cross of CCM3-flox/flox mice with Cdh5(PAC)-CreERT2 mice, to obtain tamoxifen-inducible endothelial-cell-specific expression of Cre-recombinase and CCM3 gene recombination (CCM3-ECKO mice). These mice were then crossed with BAT-gal reporter mice (16), which show βeta-catenin-activated expression of nuclear β-galactosidase (β-gal).

As reported in FIG. 7a (upper panels) the inventors could observe a significantly higher β-catenin transcription signal in the nuclei of endothelial cells in CCM3-ECKO mice in comparison to matched controls. This difference was detectable since early stages (3dpn) after induction of CCM3 recombination (at 1dpn). In brain sections of CCM3-ECKO mice endothelial cells with J3-gal-positive nuclei could be found both in pseudo-normal vessels and in cavernae of any size (FIG. 8). In contrast, phospho-Smad1 (p-Smad1) staining, in separate sections (FIG. 7a lower panels) and in co-staining for β-gal (FIG. 7b) was not enhanced after CCM3 ablation in 3dpn pups, but it was increased in 9dpn pups (FIG. 7c). Similar results were obtained for p-Smad3 (not shown). P-Smad1 was significantly high in middle-large size lesions only (maximal diameter 50 μm in 9dpn pups) (FIG. 8).

This result strongly support the pharmacological targeting of β-catenin signaling for the inhibition of initiation of new lesions. The appearance of de-novo lesions is a specific feature of the familiar form of CCM in patients. In vitro data on regression of mutated phenotype using sulindac sulfide and sulindac sulfone to inhibit β-catenin signaling are shown in FIG. 4 and FIG. 19.

### Correlation Between β-Catenin Signaling and Expression of EndMT Markers Analyzing In Vivo the Kinetics of the Two Processes in Endothelial Cells of Brain Cavernomas of CCM3-ECKO Mice

Expression of stem-cell/EndMT markers (Klf4, Ly6a, S100a4 and Id1) was high in 3dpn CCM3-ECKO pups (FIG. 9a-d, and e, single positive) and was concentrated in endothelial cells with β-gal positive nuclei (FIG. 9e, co-localization). At 9dpn β-gal expression in endothelial cells of CCM3-ECKO pups decreased while stem-cell/EndMT markers remained high (FIG. 9e). The role of β-catenin signaling in established lesions is under scrutiny, at the present as discussed in the previous paragraph

A Canonical Target of Activated β-Catenin-Driven Transcription, Axin2, is Indeed Enhanced in Endothelial Cells in Culture after Ablation of CCM1 and CCM2 Genes Besides CCM3 (FIG. 10), as Well as EndMT Markers.

Analyzing in details CCM1 KO endothelial cells in culture, the inventors observed nuclear localization of active β-catenin (dephosphorylated on Ser37 and Thr41 and escaping proteosomal degradation FIG. 11a), as already reported in CCM3-KO endothelial cells (FIG. 2). In addition, such nuclear β-catenin is transcriptionally active as indicated by the two fold-increase expression of the exogenous luciferase gene as measured in Top/Fop Flash reporter assays (a measure of activation of β-catenin/Tcf/Lef-dependent transcription; FIG. 11b). Most importantly, inhibition of β-catenin signaling using sulindac sulfone (exisulind) inhibits the expression of EndMT markers in CCM1 KO endothelial cells in culture (FIG. 11c). These data supports the concept that mutation of any CCM gene induces β-catenin signaling in association to acquisition of de-differentiated phenotype (expression of EndMT markers, FIG. 11).

In addition, sulindac sulfone (exisulind) induces re-organization of endothelial cell-to-cell contacts in CCM1 KO endothelial cells in culture, as reported for CCM3 KO endothelial cells in FIG. 3. Similar results were also obtained in CCM2 KO models.

Mechanism Through which β-Catenin Signaling is Activated in Response to CCM Ablation in Endothelial Cells

Activation of β-catenin-mediated transcription in CCM3-knockout endothelial cells was cell-autonomous since: a) it was observed in absence of exogenous Wnt; b) the porcupine inhibitors IWP2 and IWP12(26, 27) that inhibit ligand production, as well as Dkk1, an inhibitor of ligand-receptor interaction that is a competitor of Wnt co-receptor Lrp5/6 (26, 27), did not inhibit transcription of typical β-catenin (FIG. 12a-d); c) Lrp6 phosphorylation was not increased (FIGS. 12e and f); d) stimulation by exogenous Wnt3a did not induce expression of stem-cell/EndMT markers, while the constitutively active form of β-catenin, Lef-Δ13CTA(28), did (FIGS. 12g and h).

Taken together these data indicate that in CCM3-knockout endothelial cells enhanced nuclear localization and transcription activity of β-catenin do not depend on a classical ligand-receptor interaction.

The inventors reported previously that silencing or dismantling of VE-cadherin from endothelial junctions can up-regulate β-catenin signaling(18). Consistently, the inventors observed here that silencing VE-cadherin by siRNA activated the expression of EndMT markers (S100a4 and Id1) besides typical β-catenin targets (Axin2, Ccnd1 and Nkd1, FIG. 12i) and promoted nuclear localization of active β-catenin (FIG. 13a). On the contrary, VE-cadherin knockdown did not enhance the phosphorylation of Smad1 (FIG. 13b).

These data suggest that in CCM the first trigger of β-catenin signaling is the dismantling of VE-cadherin junctions that, in turn, causes the release of β-catenin in the cytoplasm and nuclear translocation. This process precedes and possibly contributes to the activation of TGF-β/BMP signaling for lesion progression.

Interestingly, dismantling of cell-to-cell junction in endothelial cells represents a common feature of the brain cavernomas induced by ablation of any CCM gene, as reported for CCM1-ECKO, CCM2-ECKO and CCM3-ECKO (FIG. 14). The inventors have previously reported disorganization of VE-cadherin in brain cavernomas of CCM1 patients (Lampugnani et al, 2010). Therefore, disorganization of junctional VE-cadherin appears a general feature of endothelial cells lining vascular cavernous malformations both in murine models and in patients. This may be accompanied by dissociation of β-catenin from VE-cadherin with enhanced nuclear translocation and increased β-catenin driven transcription also in patients, as reported above in experimental murine models. At the present, a direct proof of the activation of β-catenin signaling in brain cavernomas in patients is very difficult due to technical limitations in detecting markers of such activation in human samples. In summary, these results support the use of anti-β-catenin compounds for treatment of pathologies characterized by vascular malformation, in particular CCM patients.

Here, the inventors report that endothelial-cell-selective deletion of the CCM3 gene activates b-catenin transcription signaling in vivo in brain endothelial cells. Pharmacological inhibition of b-catenin transcriptional activity with the NSAIDs sulindac sulfide and sulindac sulfone reduces the number and dimension of cerebral and retinal vascular malformations in this murine model suggesting that b-catenin transcription signaling in endothelial cells contributes to the pathogenesis of CCM3-mediated vascular lesions.

CCM malformations develop largely, although not exclusively, in the central nervous systems in patients and in mouse models^{6,8}. Consistent with a critical role for deregulated b-catenin signaling in the endothelium in CCM pathology, the canonical Wnt pathway is a well-established determinant for the specification of the phenotype of endothelial cells at the blood-brain barrier¹¹⁻¹³. However, Wnt signaling must be abrogated postnatally to avoid abnormal vascular proliferation and morphogenesis in the central nervous system¹¹⁻¹³. In a murine model of endothelial specific b-catenin-gain-of-function the inventors observed vascular lesions in the retina comparable to those observed here in CCM3-ECKO⁴⁰. In tumor cells, the sustained induction of canonic Wnt signaling is associated to increased growth and invasion^{18,24}. In particular, upon b-catenin-mediated transcriptional activation, carcinoma cells switch from an epithelial to a mesenchymal phenotype (EMT)^{18,24}. The inventors report here that CCM3-knockout endothelial cells undergo a similar change in phenotype and upregulate a series of genes typical of EMT/EndMT²⁰. Thus, a reasonable hypothesis is that CCM lesions originate from uncontrolled kinetics and location of b-catenin signaling in endothelial cells of brain vessels.

It has recently been reported in retina and brain endothelial cells that activation of the canonical Wnt pathway by Norrin/Frizzled4 induces development and maintenance programs through both cell-autonomous and cell-nonautonomous signaling⁴¹. This can explain local vascular phenotypes in different regions of the central nervous system⁴¹. Deregulated b-catenin signaling in CCM could be supported by either endothelial-autonomous activation of the Wnt/b-catenin pathway or by abnormal responses of mutated endothelial cells to environmental Wnt, or by a combination of the two. The inventors' data indicate that at least the first of these mechanisms appears to operate in endothelial cells in culture after ablation of the CCM3 gene. Indeed, some b-catenin targets and progenitor/EndMT markers are activated through b-catenin transcription signaling under basal conditions in the CCM3-knockout endothelial cells, as their expression is inhibited by a dominant-negative Tcf4. Glading and Ginsberg¹⁰ reported similar activation of b-catenin transcription activity in bovine aorta endothelial cells and primary human arterial endothelial cells in culture after depletion of CCM1 using RNA interference. They also reported inhibition of b-catenin signaling by CCM1 in epithelial cells, both in vitro and in vivo. In addition, they reported that the phenotype of b-catenin-driven intestinal adenomas in Apc^{Min/+} mice is exacerbated in a CCM1^{+/-} background. This implies a more general regulatory role for CCM1, and possibly CCM3, in b-catenin signaling in other cells as well as in endothelial cells. As the occurrence of intestinal and other neoplasias in familial CCM patients does not appear to be increased, the specificity of vascular and organ localization for the CCM pathology suggests that endothelial differentiation and/or local factors in the organ cooperate with Wnt/b-catenin signaling for the expression of the mutated phenotype⁴². In particular, as far as is known for CCM3, mutation of this gene in neuronal cells activates astrocytes and produces vascular lesions that resemble this pathology⁹. This thus reinforces the importance of cellular crosstalk within the neurovascular unit.

Although nuclear accumulation of active b-catenin appears to be a significant characteristic of the mutated genotype, the inventors do not have direct indications of the processes that drive the b-catenin concentration into the nucleus of the CCM3-knockout endothelial cells. In general, the issue of the molecular mechanisms that regulate nuclear accumulation of b-catenin remain virtually unknown (for review, see¹⁸). However, concomitant with the increase in concentration into the nucleus, the inventors observed that b-catenin dissociates from cell-cell junctions in both the inventors' in vitro and in vivo models of endothelial-cell-specific deletion of CCM3. Junctional b-catenin is mostly associated with VE-cadherin, the transmembrane constituent of the adherens junctions⁴³, as well as with the b-catenin destruction complex⁴⁴. In the CCM3-knockout endothelial cells, the adherens junctions are disorganized, as also observed after ablation of both CCM1^{45,46} and CCM2⁶. In addition, the b-catenin associated with VE-cadherin is reduced here, as has also been observed after ablation of CCM1¹⁰. speculate that this decreased association of b-catenin with VE-cadherin is accompanied by accumulation of active b-catenin in the nucleus. This concentration of active b-catenin into the nucleus characterizes conditions of decreased junction stability in endothelial cells, as observed in sparse endothelial cells and in VE-cadherin-knockout endothelial cells¹⁷. In both of these cases, the total amount of b-catenin is reduced, even to very low levels, although the residual active b-catenin accumulates in the nucleus. Inhibition of proteosomal degradation with ‘passive’ redistribution of active b-catenin into the nucleus appears not to be likely, as the total amount of active b-catenin actually decreases.

Of particular interest, the inventors have identified two inhibitors of b-catenin transcription signaling, sulindac sulfide and sulindac sulfone, that also inhibit the development of vascular lesions in CCM3-ECKO mice. These agents are both NSAIDs that have significant chemopreventive efficacies against colon cancer in human patients, and they are under evaluation in experimental models of other types of cancer⁴⁷⁻⁵². Sulindac sulfone is potentially more interesting than the Sulindac sulfide for therapy of pathologies characterized by vascular malformation such as CCM since it lacks anti-platelet activity. The relative efficacy of sulindac sulfide versus sulindac sulfone varies depending on the type of cancer^{33,53-56}. In conclusion, the targeting of b-catenin signaling in endothelial cells with specific pharmacological tools represents an effective strategy for the reduction of vascular malformations and the prevention of the appearance of new vascular lesions, in particular in CCM patients.

### REFERENCES

1. Clatterbuck, R. E., et al. Journal of neurology, neurosurgery, and psychiatry 71, 188-192 (2001).
2. Wong, J. H., et al., Neurosurgery 46, 1454-1459 (2000).
3. Rigamonti, D., et al. The New England journal of medicine 319, 343-347 (1988).
4. Li, D. Y. & Whitehead, K. J. Stroke 41, S92-94 (2010).
5. Plummer, N. W., et al., Current neurology and neuroscience reports 5, 391-396 (2005).
6. Boulday, G., et al. The Journal of experimental medicine 208, 1835-1847 (2011).
7. Chan, A. G., et al. J Clin Invest 121, 1871-1881 (2011).
8. He, Y., et al. Science signaling 3, ra26 (2010).
9. Louvi, A., et al. Proc Natl Acad Sci USA 108, 3737-3742 (2011).
10. Glading, A. J. & Ginsberg, M. H. Disease models & mechanisms 3, 73-83 (2010).
11. Stenman, J. M., et al. Science 322, 1247-1250 (2008).
12. Daneman, R., et al. Proc Natl Acad Sci USA 106, 641-646 (2009).
13. Liebner, S., et al. The Journal of cell biology 183, 409-417 (2008).
14. Wang, Y., et al. Nature 465, 483-486 (2010).
15. Maretto, S., et al. Proc Natl Acad Sci USA 100, 3299-3304 (2003).
16. van Noort, M., Meeldijk, J., van der Zee, R., Destree, O. & Clevers, H. J Biol Chem 277, 17901-17905 (2002).
17. Taddei, A., et al. Nature cell biology 10, 923-934 (2008).
18. Clevers, H. & Nusse, R. Cell 149, 1192-1205 (2012).
19. Fadini, G. P., Losordo, D. & Dimmeler, S. Circ Res 110, 624-637 (2012).
20. Medici, D. & Kalluri, Semin Cancer Biol 22, 379-384 (2012).
21. Kovacic, J. C., et al., Circulation 125, 1795-1808 (2012).
22. Sanchez-Tillo, E., et al. Proc Natl Acad Sci USA 108, 19204-19209 (2011).
23. Li, J. & Zhou, B. P. BMC Cancer 11, 49 (2011).
24. Holland, J. D., et al., Curr Opin Cell Biol (2013).
25. Li, X., et al. Microvasc Res 84, 270-277 (2012).
26. Margariti, A., et al. Proc Natl Acad Sci USA 109, 13793-13798 (2012).
27. van Solingen, C., et al. Cardiovasc Res 92, 449-455 (2011).
28. Liang, S. X., et al. J Thromb Haemost 9, 1628-1637 (2011).
29. Stein, U., et al. Gastroenterology 131, 1486-1500 (2006).
30. James, D., et al. Nat Biotechnol 28, 161-166 (2010).
31. Tobin, N. P., et al., BMC Cancer 11, 417 (2011).
32. Kundu, J. K., et al., Biochimica et biophysica acta 1765, 14-24 (2006).
33. Thompson, H. J., et al. Cancer research 57, 267-271 (1997).
34. Boon, E. M., et al. British journal of cancer 90, 224-229 (2004).
35. Rajamanickam, S., et al., Pharmaceutical research 26, 2558-2567 (2009).
36. Prasad, C. P., et al., Chemico-biological interactions 181, 263-271 (2009).
37. Hope, C., et al. Molecular nutrition & food research 52 Suppl 1, S52-61 (2008).
38. Teiten, M. H., et al., Current pharmaceutical biotechnology 13, 245-254 (2012).
39. Lu, D., et al. Proc Natl Acad Sci USA 108, 13253-13257 (2011).
40. Corada, M., et al. Dev Cell 18, 938-949 (2010).
41. Wang, Y., et al. Cell 151, 1332-1344 (2012).
42. Wray, J. & Hartmann, C. Trends Cell Biol 22, 159-168 (2012).
43. Lampugnani, M. G., et al. The Journal of cell biology 118, 1511-1522 (1992).
44. Maher, M. T., et al., The Journal of cell biology 186, 219-228 (2009).
45. Glading, A., et al., The Journal of cell biology 179, 247-254 (2007).
46. Lampugnani, M. G., et al. Journal of cell science 123, 1073-1080 (2010).
47. Meyskens, F. L., Jr., et al. Cancer prevention research 1, 32-38 (2008).
48. Huang, L., et al. Cancer research 71, 7617-7627 (2011).
49. Takayama, T., et al. Clinical cancer research: an official journal of the American Association for Cancer Research 17, 3803-3811 (2011).
50. Steinert, G., et al. PloS one 6, e22540 (2011).
51. Giardiello, F. M., et al. The New England journal of medicine 328, 1313-1316 (1993).
52. Smalley, W., et al., Archives of internal medicine 159, 161-166 (1999).
53. Chang, W. C., et al. Annals of the New York Academy of Sciences 1059, 41-55 (2005).
54. Piazza, G. A., et al. Cancer research 57, 2909-2915 (1997).
55. Mahmoud, N. N., et al. Carcinogenesis 19, 87-91 (1998).
56. Beazer-Barclay, Y., et al. Carcinogenesis 17, 1757-1760 (1996).
57. Mackenzie, G. G., et al. Cancer prevention research 4, 1052-1060 (2011).
58. Srinivas, S., et al. BMC developmental biology 1, 4 (2001).
59. Cattelino, A., et al. The Journal of cell biology 162, 1111-1122 (2003).
60. Balconi, G., Spagnuolo, R. & Dejana, E. Arteriosclerosis, thrombosis, and vascular biology 20, 1443-1451(2000).
61. Lluis, F., et al. Cell Stem Cell 3, 493-507 (2008).
62. Zhang, N., et al. Cancer Cell 20, 427-442 (2011).
63. McDonald, D. A., et al. Stroke 43, 571-574 (2012).
Maddaluno L, et al. Nature 498, 492-496 (2013).
Vleminckx K, et al. Mechanisms of development 81:65-74 (1999).
Kundu J K et al. Biochimica et Biophysica Acta 1765 (2006): 14-24.
Anastas and Moon, 2013, Nature Rev-Cancer, 13, 11-26;
Rosenbluh et al, 2014, Trends Pharmac. Sci 35, 103-109;
Takahashi-Yanaga and Kahn, 2010, Clin Cancer Res 16, 3153-3162.
McDonald D A et al. Stroke 43, 571-574. 2012.
Dodge, Annu Rev Pharmacol Toxicol. 2011; 51:289-310.
Chen, Am J Physiol Gastrointest Liver Physiol. 2010 August; 299(2):G293-300.
Barker Nat Rev Drug Discov. 2006 December; 5(12):997-1014.
Chen et al. Nat Chem Biol 2009; 5(2):100-107.
Dodge et al J Biol Chem 2012; 287(27):23246
Wang et al J Med Chem 2013 Mar. 28; 56(6):2700-4.
Proffitt et al Cancer Res 2013 January 15; 73(2):502-7
Morrell et al PLoS One 2008 Aug. 13; 3(8):e2930.
Cruciat et al Science 2010 Jan. 22; 327(5964):459-63.
Huang et al Nature 2009 Oct. 1; 461(7264):614-20.
Thorne et al Nat Chem Biol 2010 November; 6(11):829-36.
Shan et al Biochemistry 2005 Nov. 29; 44(47):15495-503.
Park et al Biochem Biophys Res Commun 2005 Mar. 4; 328(1):227-34.
Emami et al PNAS 2004 Aug. 24; 101(34):12682-7.
Lepourcelet et al Cancer Cell 2004 January; 5(1):91-102.
Gurney et al, Proc Natl Acad Sci USA. 2012 Jul. 17; 109(29):11717-22
Medici D et al. Nature Medicine 16:1400, 2010
Medici D and Kalluri R Semin Cancer Biol 22:379, 2012
Hwang et al, 2013, Int J. Oncol 43, 261-270
Matsubayashi et al Mol Cell Biol 2004 March; 24(5):2012-24.
Gong et al Mol Cell Biol 2004 March; 24(5):2000-11.
Lu et al Cell 2004 Oct. 1; 119(1):97-108.
Cadigan et al Cell 1998 May 29; 93(5):767-777.
Glinka et al Nature 1998 Jan. 22; 391(6665):357-62.
Wallingford et al Nature 2000 May 4; 405(6782):81-5.
Zeng et al Cell 1997 Jul. 11; 90(1):181-92.
Itoh et al Curr Biol 1998 May 7; 8(10):591-4.
Willert et al Development 1999 September; 126(18):4165-73.
He et al Nature 1995 Apr. 13; 374(6523):617-22
Molenaar et al Cell 1996 Aug. 9; 86(3):391-9.
Trosset et al Proteins: structure, function and bioninformatics 64(1):60-67, 2006
Barker et al Nature Reviews Drug Discovery 5:997-1014 (2006).
Schuierer et al World J Gastroenterol 12(10):1529-1535 (2006).
Nakamura et al Genes Cells 3:395-403 (1998).
Yao et al Oncogene 24:1607-1614 (2005).
Tago et al Genes Dev. 14:1741-1749 (2000).
Green et al J Surg. Res. 101(1):16-20 (2001)
Veeramachaneni et al J. Thoracic Cardiovasc. Surg. 127(1):92-98 (2004).
Verma et al Clin. Cancer Res. 9(4):1291-300 (2003)
Gadue et al Proc. Natl. Acad. Sci. USA 103(45):16806-16811
Corada M et al. Nature Comm 4:2609, 2013
Ridder et al. J Exp Med 208 (13):2615, 2011
Whitt et al, 2012, Cancer Prev. Res. 5, 822-833
Davis et al, 2010, Nature 464, 1067-1071
Dashi et al, 2012 Adv Mater 24, 3864-3869
Cheng et al, 2013, Int J. of Oncology 43, 895-902
Fadini et al, 2012, Circ Res. 110, 624-637
Margariti A, et al, 2012, PNAS 109, 13793-13798
Li et al. 2012 Microvascular Res. 84, 270-277
Liang et al, 2011 Journal of thrombosis and haemostasis: JTH 9, 1628-1637
Stein et al. 2006 Gastroenterology, 131, 1486-1500
Medici D & Kalluri R, 2012 Seminars in cancer biology 22, 379-384
Tinsley et al, 2011 Cancer Prev Res 4, 1275-1284
Thompson et al, 2000 Cancer Res 60, 3338-3342
Liu et al, 2001 J Pharm Exp Ther 299, 583-592

1. A method for the treatment of a pathology characterized by vascular malformation, comprising administering an effective amount of an inhibitor of Wnt/β-catenin signaling to a patient in need thereof.

2. The method according to claim 1, wherein the inhibitor is a β-catenin inhibitor, in particular an inhibitor of β-catenin transcriptional signaling and/or an inhibitor of β-catenin nuclear translocation.

3. The method according to claim 1, wherein the inhibitor is a small molecule inhibitor.

4. The method according to claim 3, wherein the inhibitor is a non-steroidal anti-inflammatory drug (NSAID).

5. The method according to claim 1, wherein the inhibitor is selected from the group consisting of: quercetin, ZTM000990, PKF118-310, PKF118-744, PKF115-584, PKF-222-815, CPG049090, PNU-74654, ICG-001, NSC668036, N′-[(E)-(5-methyl-2-furyl)methylidene]-2-phenoxybenzohydrazide, N′-[(E)-1-(5-methyl-2-thienyl)ethylidene]-2-phenoxyacetohydrazide, 5-[2-(5-methyl-2-furyl)ethyl]-2-(2-thienyl)-1H-indole, 2-(2-furyl)-5-[(E)-2-(5-methyl-2-furyl)ethenyl]-1H-indole, N-[(E)-(5-methyl-2-furyl)methylidene]-4-(4-pyridinyl)-8-quinolin-amine, 2-(2-furyl)-5-[2-(5-methyl-2-furyl)ethyl]-1H-indole, 7-{(2E)-2-[(5-methyl-2-furyl)methylene]hydrazino}-N-(2-phenylethyl)-5,6-dihydrobenzo[h]isoquinoline-9-carboxamide, 1-{[(E)-(5-methyl-2-furyl)methylidene]amino}-3-(4-pyridinyl)-2,4-(1H,3H)-quinazolinedione, N-(5-methyl-2-furyl)-N-(2′-phenoxy[1,1′-biphenyl]-3-yl)amine, 4-{[7-(5-methyl-2-furyl)-2-naphthyl]oxy}pyridine, N-(5-bromo-1,3,4-oxadiazol-2-yl)-4-hydroxy-2-oxo-6-phenyl-2H-pyran-3-carboxamide, 4-hydroxy-N-(5-methyl-2-furyl)-2-oxo-6-phenyl-2H-pyran-3-carbox-amide, 3-[(E)-2-(5-bromo-1,3,4-thiadiazol-2-yl)ethenyl]-4-hydroxy-6-ph-enyl-2H-pyran-2-one, N-(5-bromo-1,3,4thiadiazol-2-yl)-4-hydroxy-2-oxo-6-phenyl-2H-pyran-3-carboxamide, 5-[(3-amino-1H-1,2,4-triazol-5-yl)methyl]-3-[3-fluoro-4-(4-morpholinyl)phenyl]-1,3-oxazolidin-2-one, 4-[(3-amino-1H-1,2,4-triazol-5-yl)methyl]-1-[3-fluoro-4-(4-morpholinyl)phenyl]-2-imidazolidin one, 1-benzhydryl-4-(5-bromo-2-furoyl)piperazine, 1-benzhydryl-4-[(5-methyl-2-thienyl)carbonyl]piperazine, benzyl (2E)-2-[1-(4-methyl-2-thienyl)ethylidene]hydrazinecarboxylate, 2-(4-chlorophenyl)-6-methyl-5-(5-methyl-1,3,4-oxadiazol-2-yl)[1,3]thiazolo[3,2-b][1,2,4]triazole, N-(5-methyl-3-isoxazolyl)-N′-[(5-phenyl-1,3,4-oxadiazol-2-yl)carbonyl]urea, N-[3-(2-{[(5-chloro-2-thienyl)methyl]sulfonyl} hydrazino)-3-oxopropyl]benzenesulfonamide-5-[3-(4-phenoxyphenyl)propyl]-1,3,4-oxadiazol-2-ol, N-(3-methyl-5-isoxazolyl)-4-phenoxybenzamide, 4-hydroxy-N-(3-methyl-5-isoxazolyl)-2-oxo-6-phenoxy-2H-pyran-3-carboxamide, 2-phenoxy-N′-[(Z)-phenyl(2-thienyl)methylidene]benzo-hydrazide, 2-anilino-N′-[(Z)-2-furyl(phenyl)methylidene]benzohydrazide, 4-[(Z)-1-(3-methyl-5-isoxazolyl)-2-phenylethenyl]phenyl 2-(1-pyrrolidinyl)ethyl ether, 5-methyl-2-furaldehyde[(3Z)-2-oxo-1-(4-pyridinyl)-1,2-dihydro-3H-indol-3-ylidene]hydrazone, (2Z)—N-[(5-methyl-2-furyl)methyl]-2-[2-oxo-1-(4-pyridinyl)-1,2-dihydro-3H-indol-3-ylidene]ethanamide, (2Z)—N-[(3-methyl-5-isoxazolyl)methyl]-2-[2-oxo-1-(4-pyridinyl)-1,2-dihydro-3H-indol-3-ylidene]ethanamide, (2-chloro-1,3-thiazol-5-yl)methyl 4-(4-morpholinylsulfonyl)phenyl ether, N-(4,5-dihydronaphtho[1,2-d][1,3]thiazol-2-yl)-N-(4-phenoxybutyl)methanesulfonamide, N-(6-methoxy-4,5-dihydronaphtho[1,2-d][1,3]thiazol-2-yl)-N-[2-(1-methyl-3-phenylpropoxy)ethyl]acetamide, 4-{2-[(5-methyl-2-furyl)methoxy]benzylidene}-1-(4-pyridinylsulfonyl)piperidine, 4-{2-[(5-bromo-2-furyl)methoxy]benzylidene}-1-isonicotinoylpiperidine, N-(4,5-dihydronaphtho[1,2-d][1,3]thiazol-2-yl)-N-(4-phenylpentyl)acetamide, N-(4,5-dihydro-3H-naphtho[1,2-d]imidazol-2-yl)-N-[2-(2-phenylethoxy)ethyl]methane sulfonamide, N′-[(Z)-(5-methyl-2-furyl)(2-pyridinyl)methylidene]-2-phenoxybenzohydrazide, sulindac, sulindac sulfide, sulindac sulfone and their pharmaceutically acceptable salts, hydroxymatairesinol, hexachlorophene, a PPARγ agonist, or PPARγ-inactive analog, silibinin, milk thistle extract (cardio mariano), EGCG (epigallocatechin-3-gallate), White tea/Green tea, Sulforaphane, Resveratrol, Curcumin, Indole-3-carbinol, Ursolic acid, Docosahexanoic acid, Genistein, β-Lapachone, salinomycin, and compounds listed in Table I.

6. The method according to claim 1, wherein the inhibitor is sulindac sulfone or sulindac sulfide or sulindac or an analog or a derivative thereof.

7. The method according to claim 1, wherein the inhibitor is selected from the group consisting of: silibinin, salinomycin, EGCG (epigallocatechin-3-gallate), White tea/Green tea, Sulforaphane, Resveratrol, Curcumin, Indole-3-carbinol, Ursolic acid, Docosahexanoic acid, Genistein and β-Lapachone.

8. The method according to claim 1, wherein the Wnt/β-catenin signaling inhibitor is a protein or peptide.

9. The method according to claim 8, wherein the protein or peptide is administered directly or expressed via an administered expression system.

10. The method according to claim 8, wherein the protein or peptide is Chibby, Axin, HDPR1, ICAT, or a fusion protein comprising an LXXLL peptide, or an antibody against frizzled.

11. The method according to claim 1, wherein the Wnt/β-catenin signaling inhibitor is an antisense nucleic acid molecule.

12. The method according to claim 11, wherein the inhibitor is a full-length antisense beta-catenin construct, beta-catenin siRNA, or beta-catenin shRNA.

13. The method according to claim 11, wherein the inhibitor is expressed by a recombinant expression system suitable for administration to the subject.

14. The method according to claim 13, wherein the recombinant expression system comprises an endothelium or a brain endothelium specific promoter element and, optionally, an inducer/repressor element.

15. The method according to claim 14, wherein the inhibitor is a protein or peptide selected from the group consisting of Chibby, Axin, HDPR1, ICAT, and a fusion protein comprising an LXXLL peptide, or an antisense nucleic acid molecule selected from the group consisting of a full-length antisense beta-catenin construct, beta-catenin siRNA, or beta-catenin shRNA.

16. The method according to claim 1, wherein the inhibitor is encapsulated in nanoparticles.

17. The method according to claim 1, wherein the vascular malformation is associated with endothelial-to-mesenchymal transition.

18. The method according to claim 1, wherein the vascular malformation is localized in the central nervous system and/or in the retina vasculature.

19. The method according to claim 1, wherein the pathology is selected from the group consisting of: fibrodysplasia ossificans progressive, cardiac fibrosis, kidney fibrosis, pulmonary fibrosis and cerebral cavernous malformation.

20. The method according to claim 1 wherein the pathology is cerebral cavernous malformation.

21. The method according to claim 20, wherein the cerebral cavernous malformation is caused by loss-of-function mutations in at least one of the genes selected from the group of: CCM1 (KRIT1), CCM2 (OSM) or CCM3 (PDCD10).

22. The method according to claim 20, wherein the cerebral cavernous malformation is sporadic or familial.

23. (canceled)

24. The method according to claim 1, further comprising administering an effective amount of at least another therapeutic agent.

25. The method according to claim 24, wherein the other therapeutic agent is selected from the group consisting of: anti-oxidant, TGF-β signaling pathway inhibitors, BMP signaling pathway inhibitors, VEGF signaling pathway inhibitors, Yap signaling pathway inhibitors, statins and other inhibitors of RhoA GTPase levels or activity.

26. (canceled)

27. (canceled)
